(19) United States
(12) Patent Application Publication
(10) Pub. No.: US 2021/0228825 A1
(43) Pub. Date: Jul. 29, 2021

(54) METHODS FOR DIAGNOSING BRUGADA SYNDROME USING AN AEROSOL

(71) Applicant: InCarda Therapeutics, Inc., Newark, CA (US)

(72) Inventors: Rangachari NARASIMHAN, Saratoga, CA (US); Luiz BELARDINELLI, Palo Alto, CA (US); Carlos A. SCHULER, Kensington, CA (US)

(21) Appl. No.: 16/972,362

(22) PCT Filed: Jun. 5, 2019
(86) PCT No.: PCT/US2019/035565

§ 371 Date: Dec. 4, 2020

Related U.S. Application Data
(60) Provisional application No. 62/680,746, filed on Jun. 5, 2018.

(51) Int. Cl. A61M 15/00

(52) U.S. Cl. CPC A61M 15/0066; A61M 15/0091; A61M 2230/04; A61M 2202/064; A61M 2202/04; A61M 15/0001

(57) ABSTRACT

Disclosed herein is a method for evaluating Brugada syndrome (BrS) in a subject in need thereof. In some embodiments, the method comprises administering an aerosol of a sodium channel blocker to the subject. Also disclosed herein are compositions, unit doses, and kits for evaluating Brugada syndrome in a subject in need thereof.

### CROSS-REFERENCE

This application claims the benefit of U.S. Provisional Application No. 62/680,746, filed Jun. 5, 2018, which application is incorporated herein by reference in its entirety.

### BACKGROUND

Brugada syndrome is a potentially life-threatening heart rhythm disorder that can be causally related to SCN5A (gene that encodes cardiac sodium channel) mutations. Patients with Brugada syndrome can have an increased risk of abnormal heart rhythms from the lower chambers of the heart (e.g., ventricular arrhythmias). Brugada syndrome can be characterized by cardiac conduction abnormalities (ST-segment abnormalities in leads V1-V3 on ECG and a high risk for ventricular arrhythmias) that can result in sudden death. Brugada syndrome presents primarily during adulthood although age at diagnosis may range from infancy to late adulthood. Many patients can have Brugada syndrome without any symptoms. However, the disease can result in severe conditions, even sudden cardiac death (SCD), in many cases. There exists a need for improved and easy-to-access diagnosis methods for Brugada syndrome.

### SUMMARY

Described herein, in some aspects, is a method of evaluating a subject in need thereof comprising: administering an aerosol of a sodium channel blocker to the subject, and assessing cardiac activity of the subject, wherein the cardiac activity is indicative of Brugada syndrome.

In some cases, the aerosol comprises a microdose of the sodium channel blocker per breath. In some cases, the aerosol comprises at most about 1000 micrograms of the sodium channel blocker per breath. In some cases, the aerosol comprises at least about 10 micrograms of the sodium channel blocker per breath. In some cases, the aerosol comprises at least about 100 micrograms of the sodium channel blocker per breath. In some cases, the aerosol comprises at least about 500 micrograms of the sodium channel blocker per breath. In some cases, the aerosol comprises at most about 10 milligrams of the sodium channel blocker per breath. In some cases, the aerosol comprises at least about 20 milligrams of the sodium channel blocker per breath. In some cases, the aerosol comprises at least about 50 milligrams of the sodium channel blocker per breath. In some cases, the aerosol comprises at least about 100 milligrams of the sodium channel blocker per breath. In some cases, the aerosol comprises about 100 micrograms to about 500 micrograms of the sodium channel blocker. In some cases, the aerosol comprises liquid droplets or dry powder, or an evaporative or condensation aerosol. In some cases, the method further comprises producing the aerosol by a nebulizer, a metered dose inhaler, or a dry powder inhaler. In some cases, the nebulizer is a vibrating mesh nebulizer or a jet nebulizer. In some cases, the dry powder inhaler is an active dry powder inhaler or a passive dry powder inhaler. In some cases, the assessing cardiac activity of the subject comprises conducting an electrocardiogram (ECG) test on the subject. In some cases, the ECG test is performed with a Holter monitor. In some cases, the ECG test is a 12-lead ECG test. In some cases, the ECG test measures at least one right precordial lead. In some cases, the ECG test measures V1, V2, or V3 lead. In some cases, the administering of the sodium channel blocker elicits an ECG change in the subject. In some cases, the ECG change appears within 60 minutes of the administering of the sodium channel blocker. In some cases, the ECG change appears within 30 minutes of the administering of the sodium channel blocker. In some cases, the ECG change appears within 10 minutes of the administering of the sodium channel blocker. In some cases, the ECG change appears within 5 minutes of the administering of the sodium channel blocker. In some cases, the administering of the sodium channel blocker unmasks an ECG phenotype of Brugada syndrome in the subject. In some cases, the ECG phenotype of Brugada syndrome is a Type 1, Type 2, or Type 3 Brugada syndrome ECG pattern. In some cases, the ECG phenotype of Brugada syndrome comprises a J wave amplitude of >2 mm or 0.2 mV in more than one right precordial lead. In some cases, the Type 1 Brugada syndrome ECG pattern comprises a negative T-wave following the J wave. In some cases, the Type 1 Brugada syndrome ECG pattern comprises a coved ST-T configuration. In some cases, the Type 1 Brugada syndrome ECG pattern comprises a descending terminal portion of ST segment. In some cases, the administering of the sodium channel blocker converts a normal ECG pattern without the sodium channel blocker to a Type 1, Type 2, or Type 3 Brugada syndrome ECG phenotype in the subject. In some cases, the administering of the sodium channel blocker converts a Type 2 Brugada syndrome ECG pattern without the sodium channel blocker to a Type 1 Brugada syndrome ECG pattern in the subject. In some cases, the Type 2 Brugada syndrome ECG pattern comprises a J wave amplitude of >2 mm or 0.2 mV in more than one right precordial lead. In some cases, the Type 2 Brugada syndrome ECG pattern comprises a positive or biphasic T-wave following the J wave. In some cases, the Type 2 Brugada syndrome ECG pattern comprises a saddleback ST-T configuration. In some cases, the Type 2 Brugada syndrome ECG pattern comprises a terminal portion of ST Segment that is elevated for at least about 1 mm or 0.1 mV. In some cases, administering of the sodium channel blocker converts a Type 3 Brugada syndrome ECG pattern without the sodium channel blocker to a Type 1 Brugada syndrome ECG pattern in the subject. In some cases, the Type 3 Brugada syndrome ECG pattern comprises a J wave amplitude of >2 nm in more than one right precordial lead. In some cases, the Type 3 Brugada syndrome ECG pattern comprises a positive T-wave following the J wave. In some cases, the Type 3 Brugada syndrome ECG pattern comprises a saddleback ST-T configuration. In some cases, the Type 3 Brugada syndrome ECG pattern comprises a terminal portion of ST Segment that is elevated for less than 1 mm or 0.1 mV. In some cases, the administering of the aerosol of the sodium channel blocker is repeated at least once to confirm a presence or absence of Brugada syndrome. In some cases, the administering of the aerosol of the sodium channel blocker is repeated two to five times to confirm the presence or absence of Brugada syndrome. In some cases, the administering of the aerosol of the sodium channel blocker is performed in a hospital or a physician clinic setting. In some cases, the sodium channel blocker is a Class I antiarrhythmic agent. In some cases, the sodium channel blocker is a Class Ic anti-arrhythmic agent. In some cases, the sodium channel blocker comprises flecainide or salt thereof. In some cases, the sodium channel blocker comprises flecainide acetate. In some cases, the sodium channel blocker is selected from the group consisting of: ajmaline, pilsicainide, flecainide, procainamide, salt and solvate thereof. In some cases, the subject has one or more of the following: (a) documented ventricular fibrillation; (b) self-terminating polymorphic ventricular tachycardia; (c) a family history of sudden cardiac death; (d) coved-type ECGs in family members; (d) electrophysiologic inducibility; or (e) syncope or nocturnal agonal respiration. In some cases, the subject has one or more genetic mutations associated with Brugada syndrome. In some cases, the method further comprises performing genetic testing of the subject's genome for one or more genetic mutations associated with Brugada syndrome.

Described herein, in some aspects, is a unit dose comprising: a composition that comprises a sodium channel blocker in a microdose that is sufficient to elicit an ECG change that unmasks an ECG phenotype of Brugada syndrome in a subject, and a pharmaceutically acceptable excipient. In some cases, the composition comprises a solution. In some cases, the composition comprises an aqueous solution. In some cases, the composition comprises a non-aqueous solution. In some cases, the composition comprises a pH buffer. In some cases, the composition comprises a pH buffer selected from the group consisting of: citrate, phosphate, phthalate, acetate, and lactate. In some cases, the composition consists essentially of the sodium channel blocker and water. In some cases, the composition consists essentially of the sodium channel blocker, water, and a pH buffer. In some cases, the composition has a pH ranging from 3.5 to 8.0. In some cases, the sodium channel blocker comprises a class Ic antiarrhythmic. In some cases, the sodium channel blocker is selected from the group consisting of: ajmaline, pilsicainide, flecainide, procainamide, salt and solvate thereof. In some cases, the unit dose comprises at most about 1000 micrograms of the sodium channel blocker. In some cases, the unit dose comprises at least about 10 micrograms of the sodium channel blocker. In some cases, the unit dose comprises at least about 100 micrograms of the sodium channel blocker. In some cases, the unit dose comprises at least about 500 micrograms of the sodium channel blocker. In some cases, the unit dose comprises about 100 micrograms to about 500 micrograms of the sodium channel blocker. In some cases, the unit dose comprises a unit dose receptacle that contains the composition.

Described herein, in some aspects, is a kit comprising any of the unit doses described herein, and instructions for use of the unit dose for evaluating a presence or absence of Brugada syndrome in a subject in need thereof.

Described herein, in some aspects, is an aerosol comprising particles having a mass median aerodynamic diameter less than 10 μm, wherein the particles comprise: a sodium channel blocker in a microdose that is sufficient to elicit an ECG change that unmasks an ECG phenotype of Brugada syndrome in a subject, and a pharmaceutically acceptable excipient.

In some cases, the particles comprise a nebulized solution. In some cases, the particles comprise a nebulized aqueous solution. In some cases, the particles comprise a pH buffer. In some cases, the particles comprise a pH buffer selected from the group consisting of: citrate, phosphate, phthalate, acetate, and lactate. In some cases, the particles consist essentially of the sodium channel blocker and water. In some cases, the particles consist essentially of the sodium channel blocker, water, and a pH buffer. In some cases, the particles have a pH ranging from 3.5 to 8.0. In some cases, the sodium channel blocker comprises a class Ic antiarrhythmic. In some cases, the sodium channel blocker is selected from the group consisting of: ajmaline, pilsicainide, flecainide, procainamide, salt and solvate thereof. In some cases, the aerosol comprises at most about 1000 micrograms of the sodium channel blocker. In some cases, the aerosol comprises at least about 10 micrograms of the sodium channel blocker. In some cases, the aerosol comprises at least about 100 micrograms of the sodium channel blocker. In some cases, the aerosol comprises at least about 500 micrograms of the sodium channel blocker. In some cases, the aerosol comprises about 100 micrograms to about 500 micrograms of the sodium channel blocker.

Described herein, in some aspects, is a kit that comprises: a container containing a sodium channel blocker in a microdose that is sufficient to elicit an ECG change that unmasks an ECG phenotype of Brugada syndrome in a subject; and an aerosolization device.

In some cases, the aerosolization device comprises a nebulizer. In some cases, the aerosolization device comprises a vibrating mesh nebulizer or a jet nebulizer. In some cases, the aerosolization device comprises a dry powder inhaler. In some cases, the aerosolization device is comprises an active dry powder inhaler or a passive dry powder inhaler. In some cases, the aerosolization device comprises a metered dose inhaler. In some cases, the sodium channel blocker comprises a class Ic antiarrhythmic. In some cases, the sodium channel blocker is selected from the group consisting of ajmaline, pilsicainide, flecainide, procainamide, salt and solvate thereof. In some cases, the container comprises at most about 1000 micrograms of the sodium channel blocker. In some cases, the container comprises at least about 10 micrograms of the sodium channel blocker. In some cases, the container comprises at least about 100 micrograms of the sodium channel blocker. In some cases, the container comprises at least about 500 micrograms of the sodium channel blocker. In some cases, the container comprises about 100 micrograms to about 500 micrograms of the sodium channel blocker.

Disclosed herein, in some aspects, are methods for diagnosing Brugada syndrome (BrS) in a patient, comprising: administering a microdose of a sodium channel blocker as an aerosol to the patient. In some cases, the aerosol is a liquid, a dry powder, a metered dose for an inhaler, an evaporative, or a condensation aerosol. In some cases, the microdose of the sodium channel blocker is at least about 10 micrograms in a single inhalation or multiple inhalations. For example, the microdose of the sodium channel blocker is at least about 10, 15, 20, 25, 30, 35, 40, 45, 50, 55, 60, 65, 70, 75, 80, 85, 90, 95, 100, 125, 150, 175, 200, 225, or 250 micrograms in a single inhalation or multiple inhalations. In some cases, the microdose of the sodium channel blocker is up to 1000 micrograms in a single inhalation or multiple inhalations. For example, the microdose of the sodium channel blocker is up to 100, 125, 150, 175, 200, 225, 250, 275, 300, 350, 400, 450, 500, 550, 600, 650, 700, 750, 800, 850, 900, 950, or 1,000 micrograms in a single inhalation or multiple inhalations. In some cases, the microdose of the sodium channel blocker is delivered in 1, 2, 3, 4, 5, 6, 7, 8, 9, or 10 inhalations. In some other cases, the microdose is at the order of milligrams, for instance, around 1, 10, 20, 50, 100, 150, 200 milligrams. The doses (micrograms or milligrams) will depend on the diagnostic agent, but given in small increments (microdoses).

In some cases, the microdose of the sodium channel blocker elicits an ECG change. In some cases, the microdose of the sodium channel blocker unmasks an ECG phenotype of Brugada syndrome. In some cases, the ECG phenotype of Brugada syndrome is a Type 1, Type 2, or Type 3 Brugada syndrome. In some cases, the microdose of the sodium channel blocker elicits an ECG change that prolongs a QRS interval. In some cases, the QRS interval is prolonged in a matter that a J-wave amplitude is >2 mm. In some cases, the microdose of the sodium channel blocker elicits an ECG change on a T-wave morphology. In some cases, the microdose of the sodium channel blocker elicits an ECG change on a ST-T wave configuration. In some cases, the ST-T wave configuration is coved shaped or saddleback shaped. In some cases, the microdose of the sodium channel blocker elicits an ECG change on a ST segment terminal portion. In some cases, the ST segment terminal portion is gradually descending, elevated to be <1 mm, or elevated to be >1 mm. In some cases, the delivering of the microdose of the sodium channel blocker is repeated at least once to confirm the presence or absence of Brugada syndrome. In some cases, the delivering of the microdose of the sodium channel blocker is repeated at least 2, 3, 4, 5, 6, 7, 8, 9, or 10 times to unmask the electrocardiographic phenotype of Brugada syndrome and /or to confirm the presence or absence of Brugada syndrome. In some cases, the delivering of the microdose of the sodium channel blocker is repeated two to five times to confirm the presence or absence of Brugada syndrome. In some cases, the administering a microdose of a sodium channel blocker is done in a hospital or physician clinic setting as an outpatient.

In some cases, the sodium channel blocker is a Class I anti-arrhythmic sodium channel blocker. In some cases, the sodium channel blocker is a Class Ia, Ib, or Ic anti-arrhythmic sodium channel blocker. In some cases, the sodium channel blocker is a Class Ic anti-arrhythmic sodium channel blocker. In some cases, the sodium channel blocker is flecainide. In some cases, the sodium channel blocker is ajmaline. In some cases, the sodium channel blocker is pilsicainide.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will he obtained by reference to the following detailed description that sets forth illustrative embodiments, in which the principles of the invention are utilized, and the accompanying drawings of which:

FIG. 1 shows the ECG phenotype of type 1 Brugada.

FIG. 2 shows how prior intravenous drug passes through the heart and lungs before reaching coronary arteries, hence coronary circulation.

FIG. 3 shows how inhaled drug of the present invention passes through the pulmonary vein to the left atrium.

FIG. 4 shows how inhaled drug of the present invention passes through directly from the lungs to the left atrium, left ventricle and then into the coronary arteries.

FIG. 5A shows the mean venous plasma concentration-time curve following administration of flecainide acetate solution by IV (2 mg/kg). Data points represent the mean±SD.

FIG. 5B shows mean venous plasma concentration-time curves following administration of flecainide acetate solution by inhalation (IH; 30 mg eTLD) or IV (2 mg/kg). Data points represent the mean±SD.

FIG. 6 shows the results of a simulation comparing intravenous and pulmonary delivery of verapamil.

### DETAILED DESCRIPTION

It is to be understood that unless otherwise indicated the present invention is not limited to dose administered, specific formulation components, drug delivery systems, manufacturing techniques, administration steps, or the like, as such may vary. In this regard, unless otherwise stated, a reference to a compound or component includes the compound or component by itself, as well as the compound or component in combination with other compounds or components, such as mixtures of compounds.

The present disclosure relates to methods, compositions, unit doses, kits, and systems that are useful for diagnosis of Brugada syndrome in a subject in need thereof. In aspects, the present disclosure relates to administration of an aerosol of sodium channel blocker into a subject in need of diagnosis. In some cases, changes in cardiac activity of the subject induced by the administration of the aerosol of sodium channel blocker are indicative of Brugada syndrome. Evaluation of Brugada syndrome can be made by monitoring the cardiac activity of the subject before, during, and after the aerosol administration. In some cases, diagnosis of Brugada syndrome in the subject is reached based on such evaluation together with other information of the subject.

Brugada Syndrome (BrS) is a genetic disease (autosomal dominant). In some cases, its diagnoses are based on genetic testing, family history, electrocardiogram (ECG) abnormalities and/or symptoms (e.g., syncope, aborted cardiac arrest). Brugada syndrome is a primary electrical disease that can be characterized by an increased risk of malignant arrhythmias and sudden cardiac death (SCD). It can cause Sudden Unexpected Death Syndrome (SUDS) or Sudden Adult Death Syndrome (SADS). Patients with Brugada Syndrome may have a normal ECGs, that can become abnormal under numerous conditions such as fever and when exposed to sodium channel (NaCh) blockers. Sodium channel blockers can be used to unmask the electrocardiogram (ECG) phenotype, referred to as the drug test for Brugada syndrome (see below).

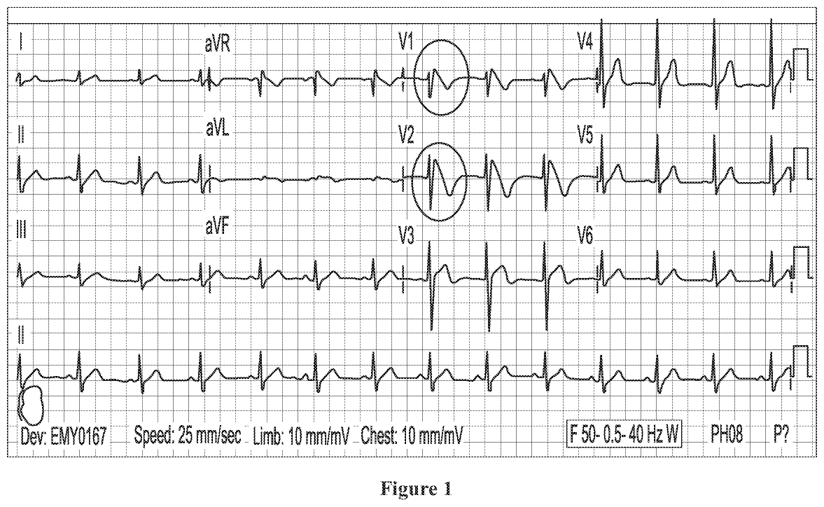

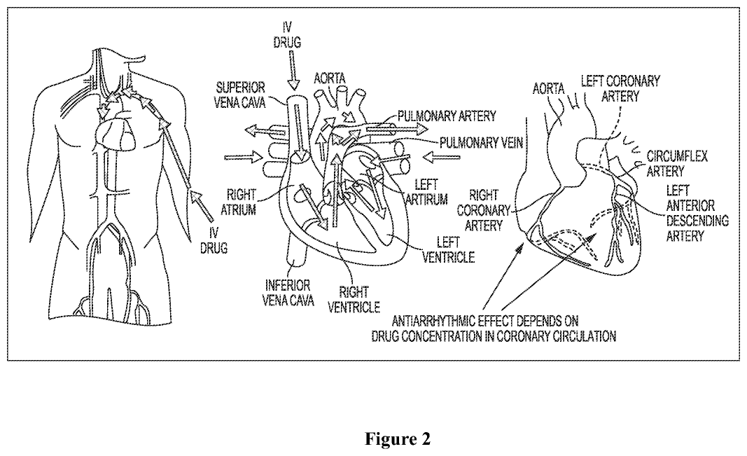

The electrocardiographic signature of Brugada Syndrome can be dynamic and can conceal ECG patterns (FIG. 1 and table 1) that can be unmasked by the administration of sodium channel blockers such as flecainide, ajmaline, procainamide and pilsicainide. Currently, drug challenge diagnoses of Brugada syndrome are mainly performed with intravenous (IV) delivery of the sodium channel blocker. When administered by IV infusion, these drugs by themselves can pose a risk of malignant arrhythmias to the patients during diagnosis, and hence can present a risk to undergo the drug test. In some cases, patients with idiopathic ventricular fibrillation also have high risk when being diagnosed as having Brugada Syndrome using intravenous delivery of sodium channel blockers. In some cases, the drugs are administered intravenously (IV) under close observation and under continuous ECG and blood pressure monitoring, and standby defibrillator. In some cases, the administration has to be immediately stopped when the ECG phenotype of Brugada syndrome is unmasked or the QRS interval is widened significantly. Nevertheless, the patient may continue to be at risk (for hours) due to the drugs being present in the systemic circulation at increased plasma concentrations sufficient to trigger life threatening ventricular tachycardia.

TABLE 1
Criteria for distinguishing each type of Brugada ECG

| | Type 1 | Type 2 | Type 3 |
|---|---|---|---|
| J - Wave | ≥2 mm | ≥2 mm | ≥2 mm |
| amplitude | | | |
| T - Wave | Negative | Positive or | Positive |
| | | biphasic | |
| ST-T | Coved | Saddleback | Saddleback |
| configuration | | | |
| ST Segment | Gradually | Elevated ≥1 mm | Elevated <1 mm |
| terminal | Descending | | |
| portion | | | |

In some aspects, disclosed herein are compositions, formulations, and methods for diagnosing patients for Brugada Syndrome. An aqueous solution can be used for the purpose of administration via inhalation in short boluses, such as microdoses, of a sodium channel blocker (e.g., flecainide, ajmaline) delivered straight to the heart and is rapidly absorbed through the lung. For example, the aqueous solution can be flecainide acetate. In some cases, the nebulization of flecainide acetate solution, ajmaline solution or a solution of any sodium channel blocker for the purpose of diagnosing patients for Brugada syndrome. The heart concentrations of flecainide can be instantly diluted in the blood stream. In some cases, the microdoses (e.g., as low as 100 micrograms) to the heart unmask the concealed ECG phenotype of Brugada syndrome. In some cases, the drug test can be expected to pose a much low overall risk to the patient compared to the same drug test administered via IV due to the very low dose delivered by simple intermittent inhalation.

As used herein, the term “microdose” can refer to a low sub-therapeutic dose of an active pharmaceutical ingredient that is unlikely to produce whole-body effects, hut in some cases, high enough to produce local or cellular effects for the purposes, such as diagnosis or research investigation, other than therapeutics. In some cases, the microdose is up to 1000 micrograms, such as no more than 900, 800, 700, 600, 500, 400, 300, 250, 200, 150, 120, 100, 80, 60, 50, 40, 30, 20, 10, 8, 6, 5, 4, 3, 2, 1 micrograms. The microdose can be a dose within any range depending on the pharmaceutical effect of the active pharmaceutical ingredient. In some cases, the microdose is at the order of milligram, up to 1000 milligrams, such as no more than 900, 800, 700, 600, 500, 400, 300, 250, 200, 150, 120, 100, 80, 60, 50, 40, 30, 20, 10, 8, 6, 5, 4, 3, 2, 1 micrograms. In some other cases, the microdose is at the order of milligrams, for instance, around 1, 10, 20, 50, 100, 150, 200 milligrams. The doses (micrograms or milligrams) will depend on the diagnostic agent, but given in small increments (microdoses).

As used herein, the singular forms “a,” “an,” and “the” can include plural referents unless the context clearly dictates otherwise. Thus, for example, reference to “a sodium channel blocker” can include not only a single active agent but also a combination or mixture of two or more different active agents.

Reference herein to “one embodiment,” “one version,” or “one aspect” can include one or more such embodiments, versions or aspects, unless otherwise clear from the context.

As used herein, the term “pharmaceutically acceptable solvate” can refer to a solvate that retains one or more of the biological activities and/or properties of the sodium channel blocker and that is not biologically or otherwise undesirable. Examples of pharmaceutically acceptable solvates include, but are not limited to, sodium channel blockers in combination with water, isopropanol, ethanol, methanol, DMSO, ethyl acetate, acetic acid, ethanolamine, or combinations thereof.

As used herein, the term “pharmaceutically acceptable salt” can refer to those salts that retain one or more of the biological activities and properties of the free acids and bases and that are not biologically or otherwise undesirable. Illustrative examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, di nitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

As used herein, “atrial arrhythmia” can refer to an arrhythmia that affects at least one atrium and does not include bradycardia. For instance, atrial arrhythmia can originate in and affect at least one atrium.

As used herein, “tachycardia” can mean an arrhythmia in which the heart rate is too fast, e.g., faster than normal. For instance, tachycardia may involve a resting heart rate of over 100 beats per minute, such as greater than 110, greater than 120, or greater than 200 beats minute.

As used herein, the term “syncope” can refer to a temporary loss of consciousness that can be related to insufficient blood flow to the brain, in some cases, syncope occurs when the heart doesn't pump enough oxygenated blood to the brain. Syncope can be related to abnormal heart rhythm (e.g., ventricular tachycardia), for instance caused by Brugada syndrome.

As used herein, the amount of an agent as described herein in the coronary circulation of the heart” can be measured by extracting a sample from any vascular region of the coronary circulation of the heart (e.g., arteries, veins, including coronary sinus) by using a cannula. The amount of the agent in the sample can then be determined by known means, such as bioanalytical techniques that employ analytical equipment such as LC-MS/MS. Thus, the amount of the agent in the blood in the heart can be measured for any particular time.

As used herein, “nominal amount” can refer to the amount contained within the unit dose receptacle(s) that are administered,

As used herein, “effective amount” can refer to an amount covering both therapeutically effective amounts and prophylactically effective amounts.

As used herein, a “therapeutically effective amount” of an active agent can refer to an amount that is effective to achieve a desired therapeutic result, and a “diagnostically effective amount” of an active agent can refer to an amount that is effective to achieve a desired diagnostic result. A therapeutically or diagnostically effective amount of a given active agent can vary with respect to factors such as the type and severity of the disorder or disease being treated or diagnosed and the age, gender, and weight of the patient. In some cases, “inhalation” (e.g., “oral inhalation” or “nasal inhalation”) refers to inhalation delivery of a therapeutically/diagnostically effective amount of a pharmaceutical agent contained in one unit dose receptacle, which, in some instance, can require one or more breaths, like 1, 2, 3, 4, 5, 6, 7, 8, 9, or more breaths. For example, if the effective amount is 90 mg, and each unit dose receptacle contains 30 mg, the delivery of the effective amount can require 3 inhalations.

As used herein, “mass median diameter” or “MMD” can refer to the median diameter of a plurality of particles, typically in a polydisperse particle population, e.g., consisting of a range of particle sizes. MMD values as reported herein are determined by laser diffraction (Sympatec Helos, Clausthal-Zellerfeld, Germany), unless the context indicates otherwise. For instance, for powders the samples are added directly to the feeder funnel of the Sympatec RODOS dry powder dispersion unit. This can be achieved manually or by agitating mechanically from the end of a VIBRI vibratory feeder element. Samples are dispersed to primary particles via application of pressurized air (2 to 3 bar), with vacuum depression (suction) maximized for a given dispersion pressure. Dispersed particles are probed with a 632.8 nm laser beam that intersects the dispersed particles' trajectory at right angles. Laser light scattered from the ensemble of particles is imaged onto a concentric array of photomultiplier detector elements using a reverse-Fourier lens assembly. Scattered light is acquired in time-slices of 5 ms. Particle size distributions are back-calculated from the scattered light spatial/intensity distribution using a proprietary algorithm.

As used herein, “geometric diameter” can refer to the diameter of a single particle, as determined by microscopy, unless the context indicates otherwise.

As used herein, “mass median aerodynamic diameter” or “MMAD” can refer to the median aerodynamic size of a plurality of particles or particles, typically in a polydisperse population. The “aerodynamic diameter” can be the diameter of a unit density sphere having the same settling velocity, generally in air, as a powder and is therefore a useful way to characterize an aerosolized powder or other dispersed particle or particle formulation in terms of its settling behavior. The aerodynamic diameter encompasses particle or particle shape, density, and physical size of the particle or particle. As used herein, MMAD refers to the median of the aerodynamic particle or particle size distribution of aerosolized particles determined by cascade impaction, unless the context indicates otherwise.

By a “pharmaceutically acceptable” component is meant a component that is not biologically or otherwise undesirable, e.g., the component may be incorporated into a pharmaceutical formulation of the invention and administered to a patient as described herein without causing any significant undesirable biological effects or interacting in a deleterious manner with any of the other components of the formulation in which it is contained. When the term “pharmaceutically acceptable” is used to refer to an excipient, it can imply that the component has met the required standards of toxicological and manufacturing testing or that it is included on the Inactive Ingredient Guide prepared by the U.S. Food and Drug Administration.

In aspects, disclosed herein is a method of evaluating a subject in need thereof comprising: administering an aerosol of a sodium channel blocker to the subject, and assessing cardiac activity of the subject. The cardiac activity can be indicative of Brugada syndrome, therefore providing basis for evaluation (e.g., diagnosis or prognosis) of Brugada syndrome in the subject. The evaluation of Brugada syndrome can include diagnosis of Brugada syndrome or prognosis of Brugada syndrome. Evaluation can be performed in a subject suspected of having Brugada syndrome or recommended for a screening for Brugada syndrome. The evaluation can be given in the form of positive or negative as a test result, or as a probability value (or in any other form) of having Brugada syndrome. Alternatively, the evaluation can be performed in a subject already diagnosed with Brugada syndrome, or a subject having received or currently receiving treatment for Brugada syndrome. The evaluation can be used a basis for prognosis for this disease.

Also disclosed is a diagnostic drug test that can be used in a hospital or physician (Cardiologist) clinic setting to unmask the ECG phenotype of Brugada syndrome and thus aid in its diagnosis. In some cases, because of the rapid decline in the concentration of the drug in the heart when administered via inhalation, the test can be done and repeated if needed to confirm the finding. In some cases, the diagnostic drug test can have advantages over current diagnostic tests including a) genetic testing requires 2-4 weeks to establish the presence or absence of known Brugada syndrome mutation which can result in erroneous false positives or be inconclusive and b) the use of IV routes of administration of sodium channel blockers which is recognized to pose significant risk of proarrhythmia to patients undergoing the drug test.

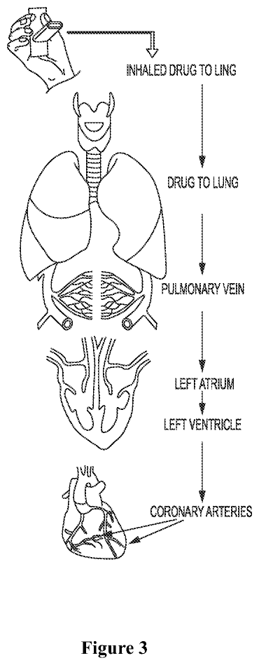

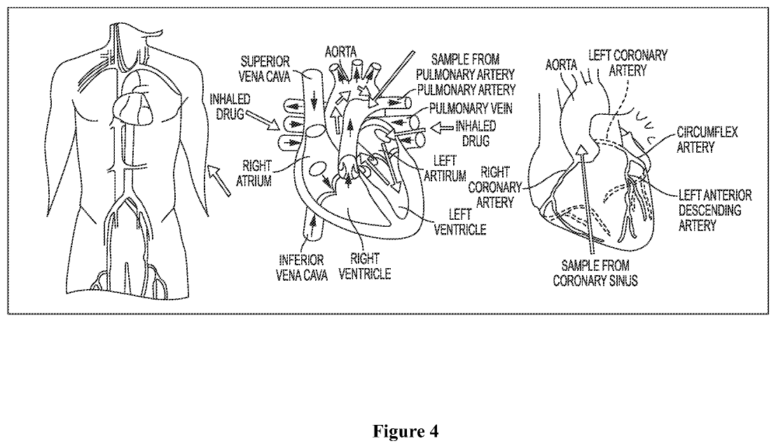

Inhalation is the shortest route for a drug to reach the heart, next only to intracardiac injection, as illustrated in FIGS. 3 and 4. Drugs delivered by inhalation generally exhibit “pulsatile pharmacokinetics” of transient high drug concentrations, followed by dilution to sub-therapeutic levels.

Inhalation can deliver sodium channel blocker, such as flecainide, in a microdose per breath, e.g., as low as 100 micrograms per breath, to the pulmonary veins to reach the ventricles, where the sodium channels can be transiently, but effectively blocked to assess beat by beat changes in the ECG with the intention to unmask the ECG phenotype of Brugada syndrome. The inhaled microdoses can be several folds lower than the IV doses as the microdoses can be delivered directly to the heart via the pulmonary route using inhalation. The advantages of inhalation can include: 1) ability to unmask the ECG phenotype of Brugada syndrome rapidly, safely, and with high sensitivity and specificity; 2) ability to reconfirm finding if needed—which cannot be done easily via IV due to the dose higher plasma concentrations and risk to patient even if in a hospital setting; and/or 3) ability to conduct in a physician's clinic as an outpatient diagnostic test, not requiring hospitalization. The pulsatile pharmacokinetic behavior of inhaled drugs show that the drug is diluted within a few seconds of reaching the heart and is diluted to safe levels in the volume of the blood. This characteristic can minimize risk to the patient. The pulsatile pharmacokinetic behavior of the drugs show that the drug is diluted within a few seconds of reaching effective concentrations in the heart and is diluted to sub-therapeutic levels in the volume of the blood. This characteristic can minimize drug-drug interactions that produce significant toxicological responses normally seen at steady state.

Thus, in certain cases, the present disclosure relates to achieving transient high drug concentrations in the heart that effect rate and rhythm changes in the heart within a short period of time allowing for unmask of conceal ECG abnormalities in patients with Brugada syndrome.

The results of the present disclosure are surprising and unexpected. In this regard, the sodium channel blocker can pass through the lungs quickly. For instance, as described in modeling work in Example 2, an antiarrhythmic agent verapamil can ionize if in salt form, so the base can pass through the lungs quickly and have a unique PK profile. In some aspects, the methods of the present disclosure take advantage of fast onset of action, high drug bioavailability, and fast absorption through the lung. Most sodium channel blockers are small molecules that have high lipid solubility and are therefore expected to have high pulmonary bioavailability and a fast rate of absorption. Another reason why the results of the present disclosure are surprising and unexpected involves the rate at which the sodium channel blocker can pass through the heart.

In some cases, the aerosol comprises a microdose of the sodium channel blocker. Such a microdose of sodium channel blocker can include up to about 1000 micrograms of sodium channel blocker, such as, no more than 900, 800, 700, 600, 500, 400, 300, 250, 200, 150, 120, 100, 80, 60, 50, 40, 30, 20, 10, 8, 6, 5, 4, 3, 2, 1 micrograms. In some cases, the microdose include at least about 10, 20, 30, 40, 50, 80, 100, 120, 150, 200, 250, 300, 350, 400, 450, 500, 600, 700, 800, or 900 micrograms of the sodium channel blocker. In some cases, the microdose include about 10, 20, 30, 40, 50, 80, 100, 120, 150, 200, 250, 300, 350, 400, 450, 500, 600, 700, 800, or 900 micrograms of the sodium channel blocker. In some other cases, the microdose is at the order of milligrams, for instance, around 1, 10, 20, 50, 100, 150, 200 milligrams of the sodium channel blocker.

The aerosol as described herein can include liquid droplets or dry powder, or an evaporative or condensation aerosol. In some cases, the aerosol is produced by an aerosolization device, such as, a nebulizer, a metered dose inhaler, or a dry powder inhaler. The administration of the aerosol can be via inhalation, such as with the aid of the aerosolization device.

In some cases, assessment of cardiac activity of the subject includes ECG test. In some cases, assessment of cardiac activity of the subject includes other test(s) that can reveal the concealed Brugada syndrome symptoms, such as electrophysiological tests, biochemistry or molecular tests. In some cases the ECG test can be performed using a Holter monitor. In some cases, genetic testing results and medical history of the patient and the patient's family members are also taken into account.

ECG test can include a standard 12-lead ECG test, In some cases, there can variations to the standard 12-lead ECG test. In a standard 12-lead ECG test, there can be six electrodes placed at chest positions, such as V I (4th intercostal space, right sternal edge), V2 (4th intercostal space, left sternal edge), V3 (midway between V2 and V4), V4 (5th intercostal space, midclavicular line), V5 (left anterior axillary line, same horizontal level as V4), and V6 (left mid-axillary line, same horizontal level as V4 & V5), and four electrode placed at limb positions, such as LA (left arm), RA (right arm), LL (left leg), and RL (right leg, neutral—not used in measurements). The 10 electrodes can produce 12 different readings (leads), including six chest leads (V1—Septal view of heart; V2—Septal view of heart; VV3—Anterior view of heart; V4—Anterior view of heart; V5—Lateral view of heart; V6—Lateral view of heart) and six other leads (Lead I—Lateral view (RA-LA); Lead II—Inferior view (RA-LL); Lead III—Inferior view (LA-LL); aVR—Lateral view (LA+LL-RA); aVL—Lateral view (RA+LL-LA); aVF—Inferior view (RA+LA-LL)).

ECG abnormalities constitute the hallmark of Brugada syndrome. They can include repolarization and depolarization abnormalities in the absence of identifiable structural cardiac abnormalities or other conditions or agents known to lead to ST-segment elevation in the right precordial leads (see Table 1). Three types of repolarization patterns are recognized (FIG. 1). Type 1 is characterized by a prominent coved ST-segment elevation displaying J wave amplitude or ST-segment elevation >=2 mm or 0.2 mV at its peak followed by a negative T-wave, with little or no isoelectric separation. Type 2 also has a high take-off ST-segment elevation, but in this case, J wave amplitude (>=2 mm) gives rise to a gradually descending ST-segment elevation (remaining >=1 mm above the baseline), followed by a positive or biphasic T-wave that results in a saddle back configuration. Type 3 is a right precordial ST-segment elevation of <1 mm of saddle back type, coved type, or both. As described herein, the terms “QRS complex,” “PR interval,” “T wave,” “ST-segment,” “ST-T configuration,” and “J wave,” as they are applied to interpret ECG recordings, are used according to their common meaning understood by a skilled artisan in cardiophysiology. In some cases, delineation of the J wave or other configurations are based on the correct placement of the precordial leads. In some cases, ECG recordings with alternative placement of the right precordial leads is possible to reveal the ECG features associated with Brugada syndrome, for instance, in individuals with high clinical suspicion (aborted sudden cardiac death victims, family members of patients with Brugada syndrome).

In some cases, the administration of the aerosol of sodium channel blocker is performed while the patient is continuously monitored, for instance, with ECG (e.g., 12 lead ECG) and blood pressure. In some cases, life support facilities are provided or close at hand, for instance, defibrillator and other advanced coronary life support facilities. In some cases, aerosol administration is stopped when the test is positive and/or when ventricular arrhythmias, such as ventricular premature complexes, are evident, or when significant QRS widening (25%) observed.

In some cases, the ECG test measures at least one right precordial lead. In some cases, the ECG test measures at least one of V1, V2, or V3 lead. The administration of the sodium channel blocker can elicit an ECG change in the subject. In some cases, the aerosol administration unmasks an ECG phenotype of Brugada syndrome in the subject. The ECG phenotype of Brugada syndrome can be Type 1, Type 2, or Type 3 Brugada syndrome ECG pattern, as described above. In some cases, the ECG phenotype of Brugada syndrome is a Type 1 Brugada syndrome ECG pattern, In some cases, the administration of the sodium channel blocker converts a normal ECG pattern without the sodium channel blocker (at baseline) to a Type 1 Brugada syndrome ECG phenotype in the subject. In some cases, the administration of the sodium channel blocker converts a normal ECG pattern to Type 2 or Type 3 Brugada syndrome ECG phenotype. In some cases, the administration of the sodium channel blocker converts a Type 2 Brugada syndrome ECG pattern without the sodium channel blocker to a Type 1 Brugada syndrome ECG pattern in the subject. In other cases, the administration of the sodium channel blocker converts a Type 3 Brugada syndrome ECG pattern without the sodium channel blocker to a Type 1 Brugada syndrome ECG pattern in the subject.

In some instance, in the case of a negative baseline ECG (e.g., normal ECG pattern), a J-wave amplitude of >2 mm absolute amplitude in lead V1 and/or V2 and/or V3 with or without RBBB is considered positive. In some cases, in patients with type 2 and type 3 ECGs, conversion of a type 2 or 3 ECG to a type 1 is considered positive for the presence of Brugada syndrome. In some cases, conversion of type 3 ECG into type 2 is considered inconclusive. In other cases, conversion of type 3 ECG into type 2 is considered positive, depending on the drug being administered to the subject and/or other parameters of concern. In some cases, ECG monitoring is continued until the ECG has normalized.

In some cases, the ECG change appears within 60 minutes of the administering of the sodium channel blocker, such as within 50, 45, 40, 35, 30, 25, 20, 15, 10, 9, 8, 7, 6, 5, 4, 3, 2, 1 minutes of the administration of the sodium channel blocker. In some cases, the administration of the aerosol of the sodium channel blocker is repeated at least once to confirm a presence or absence of Brugada syndrome, for instance, two to five times.

The sodium channel blocker as described herein can be a Class I antiarrhythmic agent, for instance, a Class Ic anti-arrhythmic agent. Non-limiting examples of the sodium channel blockers that can be used include ajmaline, pilsicainide, flecainide, procainamide, salt and solvate thereof. In some cases, flecainide acetate is used. Class Ia antiarrhythmics include, but are not limited to, quinidine, procainamide, and disopyramide, and pharmaceutically acceptable salts thereof. Class Ib antiarrhythmics include, but are not limited to, lidocaine, tocainide, phenytoin, moricizine, and mexiletine, and pharmaceutically acceptable salts thereof. Class Ic antiarrhythmics include, but are not limited to, flecainide, propafenone, and moricizine, and pharmaceutically acceptable salts thereof.

There can be criteria for selecting patients to be subject to the diagnostic method as described herein. For instance, patient that is suspected of having Brugada syndrome by physicians can be recommended for performing the evaluation as described herein. In some cases, the subject in need of the disclosed test can have one or more of the following situations: (a) documented ventricular fibrillation; (b) self-terminating polymorphic ventricular tachycardia; (c) a family history of sudden cardiac death; (d) coved-type ECGs in family members; (d) electrophysiologic inducibility; or (e) syncope or nocturnal agonal respiration. In some cases, the subject has been found having one or more genetic mutations associated with Brugada syndrome, for instance, mutation in SCN5A gene. In some cases, the diagnostic test can also include performing genetic testing of the subject's genome for one or more genetic mutations associated with Brugada syndrome. Such genetic screening/test can be performed before or after the drug challenge test as described herein.

The present disclosure also includes derivatives of the above sodium channel blockers such as solvates, salts, solvated salts, esters, amides, hydrazides, N-alkyls, and/or N-amino acyls, The derivatives of the sodium channel blockers can be pharmaceutically acceptable derivatives. Examples of ester derivatives include, but are not limited to, methyl esters, choline esters, and dimethylaminopropyl esters. Examples of amide derivatives include, but are not limited to, primary, secondary, and tertiary amides. Examples of hydrazide derivatives include, but are not limited to, N-methylpiperazine hydrazides. Examples of N-alkyl derivatives include, but are not limited to, N′,N′,N′-trimethyl and N′,N′-dimethylaminopropyl succininimidyl derivatives of sodium channel blocker methyl esters. Examples of N-aminoacyl derivatives include, but are not limited to, N-ornithyl-, N-diaminopropionyl-, N-lysil-, N-hexamethyllysil-, and N-piperdinepropionyl- or N′,N′-methyl-1-piperazine-propionyl-methyl esters.

The sodium channel blockers may exist as single stereoisomers, racemates, and/or mixtures of enantiomers, and/or diastereomers. All such single stereoisomers, racemates, and mixtures thereof are intended to be within the scope of the present invention. These various forms of the compounds may be isolated/prepared by methods known in the art.

The sodium channel blockers of the present disclosure may be prepared in a racemic mixture (e.g., mixture of isomers) that contains more than 50%, preferably at least 75%, and more preferably at least 90% of the desired isomer (e.g., 80% enantiomeric or diastereomeric excess). According to some cases, the compounds of the present invention are prepared in a form that contains at least 95% (90% e.e. or d.e.), even more preferably at least 97.5% (95% e.e. or d.e.), and most preferably at least 99% (98% e.e. or d.e.) of the desired isomer. Compounds identified herein as single stereoisomers are meant to describe compounds used in a form that contains more than 50% of a single isomer. By using known techniques, these compounds may be isolated in any of such forms by slightly varying the method of purification and/or isolation from the solvents used in the synthetic preparation of such compounds.

The pharmaceutical composition according to one or more embodiments of the present disclosure may comprise one or more sodium channel blockers and, optionally, one or more other active ingredients and, optionally, one or more pharmaceutically acceptable excipients. For example, the pharmaceutical composition may comprise neat particles of sodium channel blocker (e.g., particles containing only the sodium channel blocker), may comprise neat particles of sodium channel blocker together with other particles, and/or may comprise particles comprising sodium channel blocker and one or more active ingredients and/or one or more pharmaceutically acceptable excipients.

As noted above, the pharmaceutical composition may include one or more pharmaceutically acceptable excipient. Examples of pharmaceutically acceptable excipients include, but are not limited to, lipids, metal ions, surfactants, amino acids, carbohydrates, butters, salts, polymers, and the like, and combinations thereof.

Examples of lipids include, but are not limited to, phospholipids, glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate,

In one or more embodiments, the phospholipid comprises a saturated phospholipid, such as one or more phosphatidylcholines. Exemplary acyl chain lengths are 16:0 and 18:0 (e.g., palmitoyl and stearoyl). The phospholipid content may be determined by the active agent activity, the mode of delivery, and other factors.

Phospholipids from both natural and synthetic sources may be used in varying amounts. When phospholipids are present, the amount is typically sufficient to coat the active agent(s) with at least a single molecular layer of phospholipid. In general, the phospholipid content ranges from about 5 wt % to about 99.9 wt %, such as about 20 wt % to about 80 wt %.

Generally, compatible phospholipids can comprise those that have a gel to liquid crystal phase transition greater than about 40° C., such as greater than about 60° C., or greater than about 80° C. The incorporated phospholipids may be relatively long chain (e.g., C₁₆-C₂₂) saturated lipids. Exemplary phospholipids useful in the present invention include, but are not limited to, phosphoglycerides such as dipalmitoylphosphatidylcholine, distearoylphosphatidylcholine, diarachidoylphosphatidylcholine, dibehenoylphosphatidylcholine, diphosphatidyl glycerols, short-chain phosphatidylcholines, hydrogenated phosphatidylcholine, E-100-3 (available from Lipoid KG, Ludwigshafen, Germany), long-chain saturated phosphatidylethanolamines, long-chain saturated phosphatidylserines, long-chain saturated phosphatidylglycerols, long-chain saturated phosphatidylinositols, phosphatidic acid, phosphatidylinositol, and sphingomyelin.

Examples of metal ions include, but are not limited to, divalent cations, including calcium, magnesium, zinc, iron, and the like. For instance, when phospholipids are used, the pharmaceutical composition may also comprise a polyvalent cation, as disclosed in WO 01/85136 and WO 01/85137, which are incorporated herein by reference in their entireties. The polyvalent cation may be present in an amount effective to increase the melting temperature (Tₘ) of the phospholipid such that the pharmaceutical composition exhibits a Tₘ which is greater than its storage temperature (Tₘ) by at least about 20° C., such as at least about 40° C. The molar ratio of polyvalent cation to phospholipid may be at least about 0.05:1, such as about 0.05:1 to about 2.0:1 or about 0.25:1 to about 1.0:1. An example of the molar ratio of polyvalent cation: phospholipid is about 0.50:1. When the polyvalent cation is calcium, it may be in the form of calcium chloride. Although metal ion, such as calcium, is often included with phospholipid, none is required.

As noted above, the pharmaceutical composition may include one or more surfactants. For instance, one or more surfactants may be in the liquid phase with one or more being associated with solid particles or particles of the composition. By “associated with” it is meant that the pharmaceutical compositions may incorporate, adsorb, absorb, be coated with, or be formed by the surfactant. Surfactants include, but are not limited to, fluorinated and nonfluorinated compounds, such as saturated and unsaturated lipids, nonionic detergents, nonionic block copolymers, ionic surfactants, and combinations thereof. It should be emphasized that, in addition to the aforementioned surfactants, suitable fluorinated surfactants are compatible with the teachings herein and may be used to provide the desired preparations.

Examples of nonionic detergents include, but are not limited to, sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.), which is incorporated herein by reference in its entirety.

Examples of block copolymers include, but are not limited to, diblock and triblock copolymers of polyoxyethylene and polyoxypropylene, including poloxamer 188 (Pluronic™ F-68). poloxamer 407 (Pluronic™ F-127), and poloxamer 338.

Examples of ionic surfactants include, but are not limited to, sodium sulfosuccinate, and fatty acid soaps.

Examples of amino acids include, but are not limited to hydrophobic amino acids. Use of amino acids as pharmaceutically acceptable excipients is known in the art as disclosed in WO 95/31479, WO 96/32096, and WO 96/32149, which are incorporated herein by reference in their entireties.

Examples of carbohydrates include, but are not limited to, monosaccharides, disaccharides, and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins, and maltodextrins.

Examples of buffers include, but are not limited to, tris or citrate.

Examples of acids include, but are not limited to, carboxylic acids.

Examples of salts include, but are not limited to, sodium chloride, salts of carboxylic acids, (e.g., sodium citrate, sodium ascorbate, magnesium gluconate, sodium gluconate, tromethamine hydrochloride, etc.), ammonium carbonate, ammonium acetate, ammonium chloride, and the like.

Examples of organic solids include, but are not limited to, camphor, and the like.

The pharmaceutical composition of one or more embodiments of the present invention may also include a biocompatible, such as biodegradable polymer, copolymer, or blend or other combination thereof. In this respect useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.), Those skilled in the art will appreciate that, by selecting the appropriate polymers, the delivery efficiency of the composition and/or the stability of the dispersions may be tailored to optimize the effectiveness of the sodium channel blocker(s).

For solutions, the compositions may include one or more osmolality adjuster, such as sodium chloride. For instance, sodium chloride may be added to solutions to adjust the osmolality of the solution. In one or more embodiments, an aqueous composition consists essentially of the sodium channel blocker, the osmolality adjuster, and water.

Solutions may also comprise a buffer or a pH adjusting agent, typically a salt prepared from an organic acid or base. Representative buffers comprise organic acid salts of citric acid, lactic acid, ascorbic acid, gluconic acid, carbonic acid, tartaric acid, succinic acid, acetic acid, or phthalic acid, Tris, tromethamine hydrochloride, or phosphate buffers. Thus, the buffers include citrates, phosphates, phthalates, and lactates.

Besides the above mentioned pharmaceutically acceptable excipients, it may be desirable to add other pharmaceutically acceptable excipients to the pharmaceutical composition to improve particle rigidity, production yield, emitted dose and deposition, shelf-life, and patient acceptance. Such optional pharmaceutically acceptable excipients include, but are not limited to: coloring agents, taste masking agents, buffers, hygroscopic agents, antioxidants, and chemical stabilizers. Further, various pharmaceutically acceptable excipients may be used to provide structure and form to the particle compositions (e.g., latex particles). In this regard, it will be appreciated that the rigidifying components can be removed using a post-production technique such as selective solvent extraction.

The pharmaceutical compositions of one or more embodiments of the present invention can lack taste. In this regard, although taste masking agents are optionally included within the composition, the compositions often do not include a taste masking agent and lack taste even without a taste masking agent.

The pharmaceutical compositions may also include mixtures of pharmaceutically acceptable excipients. For instance, mixtures of carbohydrates and amino acids are within the scope of the present invention.

The compositions of one or more embodiments of the present disclosure may take various forms, such as solutions, dry powders, reconstituted powders, suspensions, or dispersions comprising a non-aqueous phase, such as propellants (e.g., chlorofluorocarbon, hydrofluoroalkane).

The solutions of the present invention are typically clear. In this regard, many of the sodium channel blockers of the present invention are water soluble.

In some cases, the isotonicity of the solution ranges from isotonic to physiologic isotonicity. Physiologic isotonicity is the isotonicity of physiological fluids.

The compositions typically have a ranging from 3.5 to 8.0, such as from 4.0 to 7.5, or 4.5 to 7.0, or 5.0 to 6.5,

For dry powders, the moisture content is typically less than about 15 wt %, such as less than about 10 wt %, less than about 5 wt %, less than about 2 wt %, less than about 1 wt %, or less than about 0.5 wt %. Such powders are described in WO 95/24183, WO 96/32149, WO 99/16419, WO 99/16420, and WO 99/16422, which are incorporated herein by reference in their entireties.

In one version, the pharmaceutical composition comprises sodium channel blocker incorporated into a phospholipid matrix. The pharmaceutical composition may comprise phospholipid matrices that incorporate the active agent and that are in the form of particles that are hollow and/or porous microstructures, as described in the aforementioned WO 99/16419, WO 99/16420, WO 99/16422, WO 01/85136, and WO 01/85137, which are incorporated herein by reference in their entireties. The hollow and/or porous microstructures are useful in delivering the sodium channel blocker to the lungs because the density, size, and aerodynamic qualities of the hollow and/or porous microstructures facilitate transport into the deep lungs during a user's inhalation. In addition, the phospholipid-based hollow and/or porous microstructures reduce the attraction forces between particles, making the pharmaceutical composition easier to deagglomerate during aerosolization and improving the flow properties of the pharmaceutical composition making it easier to process.

In one version, the pharmaceutical composition is composed of hollow and/or porous microstructures having a bulk density less than about 1.0 g/cm³, less than about 0.5 g/cm³₃, less than about 0.3 g/cm³, less than about 0.2 g/cm³, or less than about 0.1 g/cm³. By providing low bulk density particles or particles, the minimum powder mass that can be filled into a unit dose container is reduced, which eliminates the need for carrier particles. That is, the relatively low density of the powders of one or more embodiments of the present invention provides for the reproducible administration of relatively low dose pharmaceutical compounds. Moreover, the elimination of carrier particles will potentially reduce throat deposition and any “gag” effect or coughing, because large carrier particles, e.g., lactose particles, will impact the throat and upper airways due to their size.

In some aspects, the present invention involves high rugosity particles. For instance, the particles may have a rugosity of greater than 2, such as greater than 3, or greater than 4, and the rugosity may range from 2 to 15, such as 3 to 10.

In one version, the pharmaceutical composition is in dry powder form and is contained within a unit dose receptacle which may be inserted into or near the aerosolization apparatus to aerosolize the unit dose of the pharmaceutical composition. This version is useful in that the dry powder form may be stably stored in its unit dose receptacle for a long period of time. In some examples, pharmaceutical compositions of one or more embodiments of the present invention may be stable for at least 2 years. In some versions, no refrigeration is required to obtain stability. In other versions, reduced temperatures, e.g., at 2-8° C., may be used to prolong stable storage. In many versions, the storage stability allows aerosolization with an external power source.

it will be appreciated that the pharmaceutical compositions disclosed herein may comprise a structural matrix that exhibits, defines or comprises voids, pores, defects, hollows, spaces, interstitial spaces, apertures, perforations or holes. The absolute shape (as opposed to the morphology) of the perforated microstructure is generally not critical and any overall configuration that provides the desired characteristics is contemplated as being within the scope of the invention. Accordingly, some embodiments comprise approximately spherical shapes. However, collapsed, deformed or fractured particles are also compatible.

In one version, the sodium channel blocker is incorporated in a matrix that forms a discrete particle, and the pharmaceutical composition comprises a plurality of the discrete particles. The discrete particles may be sized so that they are effectively administered and/or so that they are available where needed. For example, for an aerosolizable pharmaceutical composition, the particles are of a size that allows the particles to be aerosolized and delivered to a user's respiratory tract during the user's inhalation.

The matrix material may comprise a hydrophobic or a partially hydrophobic material. For example, the matrix material may comprise a lipid, such as a phospholipid, and/or a hydrophobic amino acid, such as leucine or tri-leucine. Examples of phospholipid matrices are described in WO 99/16419, WO 99/16420, WO 99/16422, WO 01/85136, and WO 01/85137 and in U.S. Pat. Nos. 5,874,064; 5,855,913; 5,985,309; 6,503,480; and 7,473,433, and in U.S. Published App. No. 20040156792, all of which are incorporated herein by reference in their entireties. Examples of hydrophobic amino acid matrices are described in U.S. Pat. Nos. 6,372,258; 6,358,530; and 7,473,433, which are incorporated herein by reference in their entireties.

When phospholipids are utilized as the matrix material, the pharmaceutical composition may also comprise a polyvalent cation, as disclosed in WO 01/85136 and WO 01/85137, which are incorporated herein by reference in their entireties.

According to another embodiment, release kinetics of the composition containing sodium channel blocker(s) is controlled. According to one or more embodiments, the compositions of the present invention provide immediate release of the sodium channel blocker(s). Alternatively, the compositions of other embodiments of the present invention may be provided as non-homogeneous mixtures of active agent incorporated into a matrix material and unincorporated active agent in order to provide desirable release rates of sodium channel blocker. According to this embodiment, sodium channel blockers formulated using the emulsion-based manufacturing process of one or more embodiments of the present invention have utility in immediate release applications when administered to the respiratory tract. Rapid release is facilitated by: (a) the high specific surface area of the low density porous powders; (b) the small size of the drug crystals that are incorporated therein, and; (c) the low surface energy of the particles.

Alternatively, it may be desirable to engineer the particle matrix so that extended release of the active agent(s) is effected. This may be particularly desirable when the active agent(s) is rapidly cleared from the lungs or when sustained release is desired. For example, the nature of the phase behavior of phospholipid molecules is influenced by the nature of their chemical structure and/or preparation methods in spray-drying feedstock and drying conditions and other composition components utilized. In the case of spray-drying of active agent(s) solubilized within a small unilamellar vesicle (SUV) or multilamellar vesicle (MLV), the active agent(s) are encapsulated within multiple bilayers and are released over an extended time.

In contrast, spray-drying of a feedstock comprised of emulsion droplets and dispersed or dissolved active agent(s) in accordance with the teachings herein leads to a phospholipid matrix with less long-range order, thereby facilitating rapid release. While not being bound to any particular theory, it is believed that this is due in part to the fact that the active agent(s) are never formally encapsulated in the phospholipid, and the fact that the phospholipid is initially present on the surface of the emulsion droplets as a monolayer (not a bilayer as in the case of liposomes). The spray-dried particles prepared by the emulsion-based manufacturing process of one or more embodiments of the present invention often have a high degree of disorder. Also, the spray-dried particles typically have low surface energies, where values as low as 20 mN/m have been observed for spray-dried DSPC particles (determined by inverse gas chromatography). Small angle X-ray scattering (SAXS) studies conducted with spray-dried phospholipid particles have also shown a high degree of disorder for the lipid, with scattering peaks smeared out, and length scales extending in some instances only beyond a few nearest neighbors.

It should he noted that a matrix having a high gel to liquid crystal phase transition temperature is not sufficient in itself to achieve sustained release of the active agent(s). Having sufficient order for the bilayer structures is also important for achieving sustained release. To facilitate rapid release, an emulsion-system of high porosity (high surface area), and minimal interaction between the drug substance and phospholipid may be used. The pharmaceutical composition formation process may also include the additions of other composition components (e.g., small polymers such as Pluronic F-68; carbohydrates, salts, hydrotropes) to break the bilayer structure are also contemplated.

To achieve a sustained release, incorporation of the phospholipid in bilayer form may be used, especially if the active agent is encapsulated therein. In this case increasing the Tₘ of the phospholipid may provide benefit via incorporation of divalent counterions or cholesterol. As well, increasing the interaction between the phospholipid and drug substance via the formation of ion-pairs (negatively charged active+stearylamine, positively charged active+phosphatidylglycerol) would tend to decrease the dissolution rate. If the active is amphiphilic, surfactant/surfactant interactions may also slow active dissolution.

The addition of divalent counterions (e.g., calcium or magnesium ions) to long-chain saturated phosphatidylcholines results in an interaction between the negatively charged phosphate portion of the zwitterionic headgroup and the positively charged metal ion. This results in a displacement of water of hydration and a condensation of the packing of the phospholipid lipid headgroup and acyl chains. Further, this results in an increase in the Tₘ of the phospholipid. The decrease in headgroup hydration can have profound effects on the spreading properties of spray-dried phospholipid particles on contact with water. A fully hydrated phosphatidylcholine molecule will diffuse very slowly to a dispersed crystal via molecular diffusion through the water phase. The process is exceedingly slow because the solubility of the phospholipid in water is very low (about 10⁻¹⁰ mol/L for DPPC). Prior art attempts to overcome this phenomenon include homogenizing the crystals in the presence of the phospholipid. In this case, the high degree of shear and radius of curvature of the homogenized crystals facilitates coating of the phospholipid on the crystals. In contrast, “dry” phospholipid powders according to one or more embodiments of this invention can spread rapidly when contacted with an aqueous phase, thereby coating dispersed crystals without the need to apply high energies.

For example, upon reconstitution, the surface tension of spray-dried DSPC/Ca mixtures at the air/water interface decreases to equilibrium values (about 20 mN/m) as fast as a measurement can be taken. In contrast, liposomes of DSPC decrease the surface tension (about 50 mN/m) very little over a period of hours, and it is likely that this reduction is due to the presence of hydrolysis degradation products such as free fatty acids in the phospholipid. Single-tailed fatty acids can diffuse much more rapidly to the air/water interface than can the hydrophobic parent compound. Hence, the addition of calcium ions to phosphatidylcholines can facilitate the rapid encapsulation of crystalline drugs more rapidly and with lower applied energy.

In another version, the pharmaceutical composition comprises low density particles achieved by co-spray-drying nanocrystals with a perfluorocarbon-in-water emulsion. The nanocrystals may be formed by precipitation and may, e.g., range in size from about 45 μm to about 80 μm. Examples of perfluorocarbons include, but are not limited to, perfluorohexane, perfluorooctyl bromide, perfluorooctyl ethane, perfluorodecalin, perfluorobutyl ethane.

In accordance with the teachings herein the particles may be provided in a “dry” state. That is, in one or more embodiments, the particles will possess a moisture content that allows the powder to remain chemically and physically stable during storage at ambient or reduced temperature and remain dispersible. In this regard, there is little or no change in primary particle size, content, purity, and aerodynamic particle size distribution.

As such, the moisture content of the particles is typically less than about 10 wt %, such as less than about 6 wt %, less than about 3 wt or less than about 1 wt %. The moisture content is, at least in part, dictated by the composition and is controlled by the process conditions employed, e.g., inlet temperature, feed concentration, pump rate, and blowing agent type, concentration and post drying. Reduction in bound water leads to significant improvements in the dispersibility and flowability of phospholipid based powders, leading to the potential for highly efficient delivery of powdered lung surfactants or particle composition comprising active agent dispersed in the phospholipid. The improved dispersibility allows simple passive DPI devices to be used to effectively deliver these powders.

Yet another version of the pharmaceutical composition includes particle compositions that may comprise, or may be partially or completely coated with, charged species that prolong residence time at the point of contact or enhance penetration through mucosae. For example, anionic charges are known to favor mucoadhesion while cationic charges may be used to associate the formed particle with negatively charged bioactive agents such as genetic material. The charges may be imparted through the association or incorporation of polyanionic or polycationic materials such as polyacrylic acids, polylysine, polylactic acid, and chitosan.

In some versions, the pharmaceutical composition comprises particles having a mass median diameter less than about 20 μm, such as less than about 10 μm, less than about 7 μm, or less than about 5 μm. The particles may have a mass median aerodynamic diameter ranging from about 1 μm to about 6 μm, such as about 1.5 μm to about 5 μm, or about 2 μm to about 4 μm. If the particles are too large, a larger percentage of the particles may not reach the lungs. If the particles are too small, a larger percentage of the particles may be exhaled.

Unit doses of the pharmaceutical compositions may be placed in a container. Examples of containers include, but are not limited to, syringes, capsules, blow fill seal, blisters, vials, ampoules, or container closure systems made of metal, polymer (e.g., plastic, elastomer), glass, or the like. For instance, the vial may be a colorless Type I borosilicate glass ISO 6R 10 mL vial with a chlorobutyl rubber siliconized stopper, and rip-off type aluminum cap with colored plastic cover.

The container may be inserted into an aerosolization device. The container may be of a suitable shape, size, and material to contain the pharmaceutical composition and to provide the pharmaceutical composition in a usable condition. For example, the capsule or blister may comprise a wall which comprises a material that does not adversely react with the pharmaceutical composition. In addition, the wall may comprise a material that allows the capsule to be opened to allow the pharmaceutical composition to be aerosolized. In one version, the wall comprises one or more of gelatin, hydroxypropyl methylcellulose (HPMC), polyethyleneglycol-compounded HPMC, hydroxyproplycellulose, agar, aluminum foil, or the like. In one version, the capsule may comprise telescopically adjoining sections, as described for example in U.S. Pat. No. 4,247,066 which is incorporated herein by reference in its entirety. The size of the capsule may be selected to adequately contain the dose of the pharmaceutical composition. The sizes generally range from size 5 to size 000 with the outer diameters ranging from about 4.91 mm to 9.97 mm, the heights ranging from about 11.10 mm to about 26.14 mm, and the volumes ranging from about 0.13 mL to about 1.37 mL, respectively. Suitable capsules are available commercially from, for example, Shionogi Qualicaps Co. in Nara, Japan and Capsugel in Greenwood, S.C. After filling, a top portion may be placed over the bottom portion to form a capsule shape and to contain the powder within the capsule, as described in U.S. Pat. Nos. 4,846,876 and 6,357,490, and in WO 00/07572, which are incorporated herein by reference in their entireties. After the top portion is placed over the bottom portion, the capsule can optionally be banded.

For solutions, the amount of the composition in the unit dose typically ranges from about 0.5 ml to about 15 ml, such as about 2 ml to about 15 ml, from about 3 ml to about 10 ml, about 4 ml to about 8 ml, or about 5 ml to about 6 ml.

The compositions of the present invention may be made by any of the various methods and techniques known and available to those skilled in the art.

For instance, a solution of sodium channel blocker may be made using the following procedure. Typically, manufacturing equipment is sterilized before use. A portion of the final volume, e.g., 70%, of solvent, e.g., water for injection, may be added into a suitable container. Sodium channel blocker may then be added. The sodium channel blocker may be mixed until dissolved. Additional solvent may be added to make up the final batch volume. The batch may be filtered, e.g., through a 0.2 μm filter into a sterilized receiving vessel. Filling components may be sterilized before use in filling the batch into vials, e.g., 10 ml vials.

As an example, the above-noted sterilizing may include the following. A 5 liter type 1 glass bottle and lid may be placed in an autoclave bag and sterilized at elevated temperature, e.g., 121° C. for 15 minutes, using an autoclave. Similarly, vials may be placed into suitable racks, inserted into an autoclave bag, and sterilized at elevated temperature, e.g., 121° C. for 15 minutes, using an autoclave. Also similarly, stoppers may be placed in an autoclave bag and sterilized at elevated temperature, e.g., 121° C., for 15 minutes, using an autoclave. Before sterilization, sterilizing filters may be attached to tubing, e.g., a 2 mm length of 7 mm×13 mm silicone tubing. A filling line may be prepared by placed in an autoclave bag and sterilized at elevated temperature, e.g., 121° C. for 15 minutes, using an autoclave.

The above-noted filtration may involve filtration into a laminar flow work area. The receiving bottle and filters may be set up in the laminar flow work area.

The above-noted filling may also be conducted under laminar flow protection. The filling line may be unwrapped and placed into the receiving bottle. The sterilized vials and stoppers may be unwrapped under laminar flow protection. Each vial may be filled, e.g., to a target fill of 5 g, and stoppered. A flip off collar may be applied to each vial. The sealed vials may be inspected for vial leakage, correct overseals, and cracks.

In certain cases, the sodium channel blocker may he in a solution. In particular examples, the solution is an aqueous solution. In some examples, the sodium channel blocker can be present at a concentration in the range of about 1 microgram/mL to 10 mg/mL, such as about 1 to 10, 1 to 20, 1 to 30, 1 to 40, 1 to 50, 1 to 60, 1 to 70, 1 to 80, 1 to 90, 1 to 100, 1 to 120, 1 to 150, 1 to 180, 1 to 200, 1 to 220, 1 to 250, 1 to 280, 1 to 300, 1 to 320, 1 to 350, 1 to 380, 1 to 400, 1 to 420, 1 to 450, 1 to 480, 1 to 500, 10 to 20, 10 to 30, 10 to 40, 10 to 50, 10 to 60, 10 to 70, 10 to 80, 10 to 90, 10 to 100, 10 to 120, 10 to 150, 10 to 180, 10 to 200, 10 to 220, 10 to 250, 10 to 280, 10 to 300, 10 to 320, 10 to 350, 10 to 380, 10 to 400, 10 to 420, 10 to 450, 10 to 480, 10 to 500, 10 to 60, 50 to 70, 50 to 80, 50 to 90, 50 to 100, 50 to 120, 50 to 150, 50 to 180, 50 to 200, 50 to 220, 50 to 250, 50 to 280, 50 to 300, 50 to 320, 50 to 350, 50 to 380, 50 to 400, 50 to 420, 50 to 450, 50 to 480, 50 to 500, 100 to 120, 100 to 150, 100 to 180, 100 to 200, 100 to 220, 100 to 250, 100 to 280, 100 to 300, 100 to 320, 100 to 350, 100 to 380, 100 to 400, 100 to 420, 100 to 450, 100 to 480, 100 to 500, 200 to 220, 200 to 250, 200 to 280, 200 to 300, 200 to 320, 200 to 350, 200 to 380, 200 to 400, 200 to 420, 200 to 450, 200 to 480, 200 to 500, 200 to 600, 200 to 700, 200 to 800, 200 to 900, 200 to 1000, 300 to 400, 300 to 420, 300 to 450, 300 to 480, 300 to 500, 300 to 600, 300 to 700, 300 to 800, 300 to 900, 300 to 1000, 300 to 1200, 300 to 1500, 300 to 1800, 300 to 2000, 300 to 2500, 300 to 3000, 300 to 4000, 300 to 5000, 300 to 6000, 300 to 8000, 300 to 700, 500 to 800, 500 to 900, 500 to 1000, 500 to 1200, 500 to 1500, 500 to 1800, 500 to 2000, 500 to 2500, 500 to 5000, 500 to 4000, 500 to 5000, 500 to 6000, 500 to 8000, 1000 to 1200, 1000 to 1500, 1000 to 1800, 1000 to 2000, 1000 to 2500, 1000 to 10000, 1000 to 4000, 1000 to 5000, 1000 to 6000. 1000 to 8000, 1000 to 9000, 2000 to 2500, 2000 to 20000, 2000 to 4000, 2000 to 5000, 2000 to 6000, 2000 to 8000, 2000 to 9000, or 2000 to 10000 microgram/mL; 20 to 50 milligram/mL, or 20 to 100 milligram/mL.

As another example, a sodium channel blocker may be prepared by lyophilizing the sodium channel blocker to form a powder for storage. The powder is then reconstituted prior to use. This technique may be used when the sodium channel blocker is unstable in solution.

In some cases, the lyophilized powder can be reconstituted in a suitable solvent such that the sodium channel blocker is present at a concentration from about 1 microgram/mL to 100 mg/mL, such as about 1 to 10, 1 to 20, 1 to 30, 1 to 40, 1 to 50, 1 to 60, 1 to 70, 1 to 80, 1 to 90, 1 to 100, 1 to 120, 1 to 150, 1 to 180, 1 to 200, 1 to 220, 1 to 250, 1 to 280, 1 to 300, 1 to 320, 1 to 350, 1 to 380, 1 to 400, 1 to 420, 1 to 450, 1 to 480, 1 to 500, 10 to 20, 10 to 30, 10 to 40, 10 to 50, 10 to 60, 10 to 70, 10 to 80, 10 to 90, 10 to 100, 10 to 120, 10 to 150, 10 to 180, 10 to 200, 10 to 220, 10 to 250, 10 to 280, 10 to 300, 10 to 320, 10 to 350, 10 to 380, 10 to 400, 10 to 420, 10 to 450, 10 to 480, 10 to 500, 10 to 60, 50 to 70, 50 to 80, 50 to 90, 50 to 100, 50 to 120, 50 to 150, 50 to 180, 50 to 200, 50 to 220, 50 to 250, 50 to 280, 50 to 300, 50 to 320, 50 to 350, 50 to 380, 50 to 400, 50 to 420, 50 to 450, 50 to 480, 50 to 500, 100 to 120, 100 to 150, 100 to 180, 100 to 200, 100 to 220, 100 to 250, 100 to 280, 100 to 300, 100 to 320, 100 to 350, 100 to 380, 100 to 400, 100 to 420, 100 to 450, 100 to 480, 100 to 500, 200 to 220, 200 to 250, 200 to 280, 200 to 300, 200 to 320, 200 to 350, 200 to 380, 200 to 400, 200 to 420, 200 to 450, 200 to 480, 200 to 500, 200 to 600, 200 to 700, 200 to 800, 200 to 900, 200 to 1000, 300 to 400, 300 to 420, 300 to 450, 300 to 480, 300 to 500, 300 to 600, 300 to 700, 300 to 800, 300 to 900, 300 to 1000, 300 to 1200, 300 to 1500, 300 to 1800, 300 to 2000, 300 to 2500, 300 to 3000, 300 to 4000, 300 to 5000, 300 to 6000, 300 to 8000, 300 to 700, 500 to 800, 500 to 900, 500 to 1000, 500 to 1200, 500 to 1500, 500 to 1800, 500 to 2000, 500 to 2500, 500 to 5000, 500 to 4000, 500 to 5000, 500 to 6000, 500 to 8000, 1000 to 1200, 1000 to 1500, 1000 to 1800, 1000 to 2000, 1000 to 2500, 1000 to 10000, 1000 to 4000, 1000 to 5000, 1000 to 6000, 1000 to 8000, 1000 to 9000, 2000 to 2500, 2000 to 20000, 2000 to 4000, 2000 to 5000, 2000 to 6000, 2000 to 8000, 2000 to 9000, or 2000 to 10000 microgram/mL; 20 to 50 milligram/mL, 20 to 100 milligram/mL.

The solvent for the solution to be lyophilized may comprise water. The solution may be excipient-free. For instance, the solution may be cryoprotectant-free.

In one or more embodiments, a suitable amount (e.g., 120 g per liter of final solution) of drug substance may be dissolved, e.g., in about the 75% of the theoretical total amount of water for injection under nitrogen bubbling. The dissolution time may be recorded and appearance may be evaluated.

Then, the dilution to the final volume with WFI may be carried out. Final volume may be checked. Density, pH, endotoxin, bioburden, and content by UV may be measured both before and after sterile filtration.

The solution may be filtered before lyophilizing. For instance, a double 0.22 μm filtration may be performed before filling. The filters may be tested for integrity and bubble point before and after the filtration.

Pre-washed and autoclaved vials may be aseptically filled using an automatic filling line to a target of 5 ml per vial and then partially stoppered. In process check for fill volumes may be done by checking the fill weight every 15 minutes.

The lyophilizing is generally conducted within about 72 hours, such as within about 8 hours, or within about 4 hours, of the dissolving.

In one or more embodiments, the lyophilizing comprises freezing the solution to form a frozen solution. The frozen solution is typically held at an initial temperature ranging from about −40° C. to about −50° C., such as about −45° C. During the initial temperature period, the pressure around the frozen solution is typically atmospheric pressure. The initial temperature period typically ranges from about 1 hour to about 4 hours, such about 1.5 hours to about 3 hours, or about 2 hours.

The lyophilizing may further comprise raising a temperature of the frozen solution to a first predetermined temperature, which may range from about 10° C. to about 20° C., such as about 15° C. The time for the heat ramp from the initial temperature to the first predetermined temperature generally ranges from about 6 hours to about 10 hours, such as about 7 hours to about 9 hours.

During the first predetermined temperature period, the pressure around the solution typically ranges from about 100 μbar to about 250 μbar, such as about 150 μbar to about 225 μbar. The solution may be held at the first predetermined temperature for a period ranging from about 20 hours to about 30 hours, such as from about 24 hours.

The lyophilizing may still further comprise raising a temperature of the solution to a second predetermined temperature, which may range from about 25° C. to about 35° C., such as about 30° C. During the second predetermined temperature period, the pressure around the frozen solution typically ranges from about 100 μbar to about 250 μbar, such as about 150 μbar to about 225 μbar. The solution may be held at the second predetermined temperature for a period ranging from about 10 hours to about 20 hours.

In view of the above, the lyophilization cycle may comprise a freezing ramp, e.g., from 20° C. to −45° C. in 65 minutes, followed by a freeze soak, e.g., at −45° C. for 2 hours. Primary drying may be accomplished with a heating ramp, e.g., from −45° C. to 15° C. in 8 hours, followed by a temperature hold, e.g., at 15° C. for 24 hours at a pressure of 200 μbar. Secondary drying may be accomplished with a heating ramp, e.g., from 15° C. to 30° C. in 15 minutes, followed by a temperature hold at 30° C. for 15 hours at a pressure of 200 μbar. At the end of the lyophilization cycle, the vacuum may be broken with sterile nitrogen, and the vials may be automatically stoppered.

The water content of the lyophilized powder is typically less than about 7 wt %, such as less than about 5 wt %, less than about 4 wt %, less than about 3 wt %, less than about 2 wt %, or less than about 1 wt %.

The powder is capable of being reconstituted with water at 25′ C. and 1.0 atmosphere and with manual agitation, in less than about 60 seconds, such as less than about 30 seconds, less than about 15 seconds, or less than about 10 seconds,

The powder typically has a large specific surface area that facilitates reconstitution. The specific surface area typically ranges from about 5 m²/g to about 20 m²/g, such as about 8 m²/g to 15 m²/g, or about 10 m²/g to 12 m²/g.

Upon reconstitution with water, the sodium channel blocker solution typically has a pH that ranges from about 2.5 to about 7, such as about 3 to about 6.

For dry powders, the composition may be formed by spray drying, lyophilization, milling (e.g., wet milling, dry milling), and the like.

In spray drying, the preparation to be spray dried or feedstock can be any solution, coarse suspension, slurry, colloidal dispersion, or paste that may be atomized using the selected spray drying apparatus. In the case of insoluble agents, the feedstock may comprise a suspension as described above. Alternatively, a dilute solution and/or one or more solvents may be utilized in the feedstock, In one or more embodiments, the feed stock will comprise a colloidal system such as an emulsion, reverse emulsion microemulsion, multiple emulsion, particle dispersion, or slurry.

In one version, the sodium channel blocker and the matrix material are added to an aqueous feedstock to form a feedstock solution, suspension, or emulsion. The feedstock is then spray dried to produce dried particles comprising the matrix material and the sodium channel blocker. Suitable spray-drying processes are known in the art, for example as disclosed in WO 99/16419 and U.S. Pat. Nos. 6,077,543; 6,051,256; 6,001,336; 5,985,248; and 5,976,574, which are incorporated herein by reference in their entireties.

Whatever components are selected, the first step in particle production typically comprises feedstock preparation. If a phospholipids-based particle is intended to act as a carrier for the sodium channel blocker, the selected active agents) may be introduced into a liquid, such as water, to produce a concentrated suspension. The concentration of sodium channel blocker and optional active agents typically depends on the amount of agent required in the final powder and the performance of the delivery device employed (e.g., the fine particle dose for a metered dose inhaler (MDI) or a dry powder inhaler (DPI)).

Any additional active agent(s) may be incorporated in a single feedstock preparation and spray dried to provide a single pharmaceutical composition species comprising a plurality of active agents. Conversely, individual active agents could be added to separate stocks and spray dried separately to provide a plurality of pharmaceutical composition species with different compositions. These individual species could be added to the suspension medium or powder dispensing compartment in any desired proportion and placed in the aerosol delivery system as described below.

Polyvalent cation may be combined with the sodium channel blocker suspension, combined with the phospholipid emulsion, or combined with an oil-in-water emulsion formed in a separate vessel. The sodium channel blocker may also be dispersed directly in the emulsion.

For example, polyvalent cation and phospholipid may be homogenized in hot distilled water (e.g., 70° C.) using a suitable high shear mechanical mixer (e.g., Ultra-Turrax model T-25 mixer) at 8000 rpm for 2. to 5 min. Typically, 5 to 25 g of fluorocarbon is added dropwise to the dispersed surfactant solution while mixing. The resulting polyvalent cation-containing perfluorocarbon in water emulsion may then be processed using a high pressure homogenizer to reduce the particle size. Typically, the emulsion is processed for five discrete passes at 12,000 to 18,000 PSI and kept at about 50° C. to about 80° C.

When the polyvalent cation is combined with an oil-in-water emulsion, the dispersion stability and dispersibility of the spray dried pharmaceutical composition can be improved by using a blowing agent, as described in WO 99/16419, which is incorporated herein by reference in its entirety. This process forms an emulsion, optionally stabilized by an incorporated surfactant, typically comprising submicron droplets of water immiscible blowing agent dispersed in an aqueous continuous phase. The blowing agent may be a fluorinated compound (e.g., perfluorohexane, perfluorooctyl bromide, perfluorooctyl ethane, perfluorodecalin, perfluorobutyl ethane) which vaporizes during the spray-drying process, leaving behind generally hollow, porous aerodynamically light particles. Other suitable liquid blowing agents include non-fluorinated oils, chloroform, Freon® fluorocarbons, ethyl acetate, alcohols, hydrocarbons, nitrogen, and carbon dioxide gases. The blowing agent may be emulsified with a phospholipid.

Although the pharmaceutical compositions may be formed using a blowing agent as described above, it will be appreciated that, in some instances, no additional blowing agent is required and an aqueous dispersion of the sodium channel blocker and/or pharmaceutically acceptable excipients and surfactant(s) are spray dried directly. In such cases, the pharmaceutical composition may possess certain physicochemical properties (e.g., high crystallinity, elevated melting temperature, surface activity, etc.) that make it particularly suitable for use in such techniques.

As needed, cosurfactants such as poloxamer 188 or span 80 may be dispersed into this annex solution. Additionally, pharmaceutically acceptable excipients such as sugars and starches can also be added.

The feedstock(s) may then be fed into a spray dryer. Typically, the feedstock is sprayed into a current of warm filtered air that evaporates the solvent and conveys the dried product to a collector. The spent air is then exhausted with the solvent. Commercial spray dryers manufactured by Buchi Ltd. or Niro Corp. may be modified for use to produce the pharmaceutical composition. Examples of spray drying methods and systems suitable for making the dry powders of one or more embodiments of the present invention are disclosed in U.S. Pat. Nos. 6,077,543; 6,051,256; 6,001,336; 5,985,248; and 5,976,574, which are incorporated herein by reference in their entireties.

Operating conditions of the spray dryer such as inlet and outlet temperature, feed rate, atomization pressure, flow rate of the drying air, and nozzle configuration can be adjusted in order to produce the required particle size, and production yield of the resulting dry particles. The selection of appropriate apparatus and processing conditions are within the purview of a skilled artisan in view of the teachings herein and may be accomplished without undue experimentation. Exemplary settings are as follows: an air inlet temperature between about 60° C. and about 170° C.; an air outlet between about 40° C. to about 120° C.; a feed rate between about 3 mL/min to about 15 mL/min; an aspiration air flow of about 300 L/min; and an atomization air flow rate between about 25/min and about 50 L/min. The settings will, of course, vary depending on the type of equipment used. In any event, the use of these and similar methods allow formation of aerodynamically light particles with diameters appropriate for aerosol deposition into the lung.

Hollow and/or porous microstructures may be formed by spray drying, as disclosed in WO 99/16419, which is incorporated herein by reference. The spray-drying process can result in the formation of a pharmaceutical composition comprising particles having a relatively thin porous wall defining a large internal void. The spray-drying process is also often advantageous over other processes in that the particles formed are less likely to rupture during processing or during deagglomeration.

Pharmaceutical compositions useful in one or more embodiments of the present invention may alternatively be formed by lyophilization. Lyophilization is a freeze-drying process in which water is sublimed from the composition after it is frozen. The lyophilization process is often used because biologics and pharmaceuticals that are relatively unstable in an aqueous solution may be dried without exposure to elevated temperatures, and then stored in a dry state where there are fewer stability problems. With respect to one or more embodiments of the instant invention, such techniques are particularly compatible with the incorporation of peptides, proteins, genetic material and other natural and synthetic macromolecules in pharmaceutical compositions without compromising physiological activity. Lyophilized cake containing a fine foam-like structure can be micronized using techniques known in the art to provide particles of the desired size.

The compositions of one or more embodiments of the present invention may be administered by oral inhalation.

Moreover, the doses of composition that are inhaled are typically much less than those administered by other routes and required to obtain similar effects, due to the efficient targeting of the inhaled composition to the heart.

In one or more embodiments of the invention, a pharmaceutical composition comprising sodium channel blocker is administered to the lungs of a patient in need thereof. For example, the patient may have been diagnosed with an arrhythmia. Examples of arrhythmias include, but are not limited to, tachycardia, supraventricular tachycardia (SVT), paroxysmal supraventricular tachycardia (PSVT), atrial fibrillation (AF), paroxysmal atrial fibrillation (PAF), persistent atrial fibrillation, permanent atrial fibrillation, atrial flutter, paroxysmal atrial flutter, and lone atrial fibrillation, and ventricular tachycardia (monomorphic or polymorphic), non-sustained or sustained.

This method of diagnosis can result in effectively delivering microdoses of a Sodium channel blocker rapidly and as often as needed such that a bolus dose reaches almost instantly the heart. In Brugada syndrome, the heart can be very sensitive to these transient blocking effect of Sodium channel blockers reflected by a) widening of QRS; b) presence/appearance of the typical Brugada syndrome ECG (see FIG. 1 and Table 1); and/or c) the occurrence of premature ventricular ectopy. These ECG changes can be transient as the drug rapidly exits the heart and is diluted in systemic circulation. Due to the transient (e.g., in minutes) nature of these changes, combined with the low concentration of the drug, the test can be done more safely by delivering only the amount of drug (e.g., flecainide or ajmaline) required to unmask the ECG phenotype of Brugada syndrome. The test can also be repeated to confirm the presence or absence of Brugada syndrome if necessary. The ability to use this drug diagnostic test in patients harboring Brugada syndrome mutations should pose less risk of life threatening arrhythmias due the overall lower exposure of drug to the heart and systemic circulation e.g., AUC) compared to the IV or PO route of administration.

The time for dosing is typically short. For nebulizers the dosing time usually ranges from 15 seconds to 20 minutes, such as from 15 seconds to 15 minutes, or from 15 seconds to 10 minutes. Regarding dry powders, for a single capsule, the total dosing time is normally less than about 1 minute. Thus, the time for dosing may be less than about 5 min, such as less than about 4 min, less than about 3 min, less than about 2 min, or less than about 1 min.

The nebulization of flecainide acetate solution or ajmaline solution can be used for diagnosing Brugada Syndrome prior to implementing any therapy, pharmacological or device such as ICD implantation. It can be beneficial to have a safe test that has high sensitivity, specificity and predictive value.

The methods disclosed herein can effectively deliver microdoses of a sodium channel blocker (e.g., flecainide, ajmaline) directly to the heart via the lungs. In some cases, the effect of these microdoses can be reliably measured using a surface ECG (e.g., unmask the underlying ECG phenotype of BrS), and therefore only the amount of drug necessary for the diagnosis is administered.

When drugs are administered via inhalation, multiple inhalations can be used to achieve the desired dose. In some cases, each inhalation constitutes a microdose that reaches the heart via lung. In some cases, a microdose of as less as 100 micrograms can elicit a response in a surface ECG. The response can be in the form of a change in the ST-T and J wave configurations (e.g., saddle-back or coved shape) phenotypical of the ECG of Brugada syndrome (see Figure and Table 1). In some cases, the microdose can be diluted in the blood stream so as not to induce proarrhythmia. The response can be tailored systematically, with single to multiple breaths to deliver doses of flecainide until the ECG phenotype of Brugada syndrome is unmasked. The test can be repeated to confirm the presence of Brugada and/or also to confirm the absence of Brugada by administration of a higher dose until QRS widens. For example, FIG. 1 shows the ECG phenotype of type 1 Brugada.

In another aspect, the present invention is directed to a unit dose comprising a composition. In some cases, the unit dose further comprises a unit dose receptacle containing the composition. The composition comprises a sodium channel blocker in a microdose amount of the sodium channel blocker sufficient to elicit an ECG change that unmasks an ECG phenotype of Brugada syndrome, and a pharmaceutically acceptable excipient.

In still another aspect, the present invention is directed to an aerosol comprising particles having a mass median aerodynamic diameter less than 10 μm. The particles comprise at least one sodium channel blocker in an in a microdose amount of the sodium channel blocker sufficient to elicits an ECG change that unmasks an ECG phenotype of Brugada syndrome, and a pharmaceutically acceptable excipient.

In some cases, the pulmonary administration comprises nebulizing a solution comprising the sodium channel blocker. In some cases, the nebulizing comprises nebulizing with a vibrating mesh nebulizer. In some cases, the nebulizing comprises nebulizing with a jet nebulizer. In some cases, the nebulizing comprises nebulizing with a breath-activated nebulizer. In some cases, the nebulizing comprises nebulizing with a spray nozzle array creating Rayleigh jets. In some cases, the nebulizing comprises forming droplets having a mass median aerodynamic diameter of less than 10 μm. In some cases, the pulmonary administration comprises administering a dry powder comprising the at least one sodium channel blocker. In some cases, the dry powder comprises particles having a mass median aerodynamic diameter of less than 10 μm. In some cases, the dry powder is administered via an active dry powder inhaler. In some cases, the dry powder is administered via a passive dry powder inhaler. In some cases, the pulmonary administration comprises administering the at least one sodium channel blocker via a metered dose inhaler. In some cases, the metered dose inhaler forms particles having a mass median aerodynamic diameter of less than 10 μm. In some cases, the metered dose inhaler contains the at least one sodium channel blocker formulated in a carrier selected from hydrofluoroalkane and chlorofluorocarbon.

In another aspect, the present invention is directed to a unit dose comprising: a composition that comprises a sodium channel blocker in a microdose amount of the sodium channel blocker sufficient to elicit an ECG change that unmasks an ECG phenotype of Brugada syndrome; and a pharmaceutically acceptable excipient. In some cases, the composition comprises a solution. In some cases, the composition comprises a solution having a tonicity that ranges from isotonic to physiologic isotonicity. In some cases, the composition comprises an aqueous solution. In some cases, the composition comprises a non-aqueous solution. In some cases, the composition further comprises a pH buffer. In some cases, the composition further comprises a pH buffer selected from citrate, phosphate, phthalate, and lactate. In some cases, the composition consists essentially of the at least one sodium channel blocker and water. In some cases, the composition consists essentially of the at least one sodium channel blocker, water, and a pH buffer. In some cases, the composition has a pH ranging from 3.5 to 8.0.

Yet another aspect of the present invention relates to a kit. The kit can comprise a unit dose as described herein and instructions for use of the unit dose for evaluating a presence or absence of Brugada syndrome in a subject in need thereof. For example, and not by way of limitation, the instructions can include a description of the unit dose, the sodium channel blocker, and, optionally, other components included in the kit, and methods for administration, including methods for aerosolizing the composition (if not aerosolized yet), methods for determining the proper state of the subject, the proper dosage amount and the proper administration method for administering the sodium channel blocker. Instructions can also include guidance for monitoring the subject over duration of the test. The instructions can be provided in the form of paper sheet, or stored in optic discs, USB drive, or other transferrable computer-readable media.

In some cases, the kit comprises a container containing at least one sodium channel blocker and an aerosolization device. In some cases, the aerosolization device comprises a nebulizer. In some cases, the aerosolization device comprises a vibrating mesh nebulizer. In some cases, the aerosolization device comprises a jet nebulizer. In some cases, the aerosolization device comprises a device containing a Rayleigh jet spray nozzle. In some cases, the aerosolization device comprises a dry powder inhaler. In some cases, the aerosolization device comprises an active dry powder inhaler. In some cases, the aerosolization device comprises a passive dry powder inhaler. In some cases, the aerosolization device comprises a metered dose inhaler. In some cases, an amount of the at least one sodium channel blocker is sufficient to produce an ECG change that unmasks an ECG phenotype of Brugada syndrome.

### Exemplary Embodiments

Embodiment 1. A method for diagnosing Brugada syndrome BrS) in a patient comprising: administering a microdose of a sodium channel blocker as an aerosol to the patient.

Embodiment 2. The method of embodiment 1, wherein the aerosol is a liquid, a dry powder, a metered dose for an inhaler, an evaporative, or a condensation aerosol.

Embodiment 3. The method of embodiment 1 or 2, wherein the microdose of the sodium channel blocker is at least about 10 micrograms in a single inhalation or multiple inhalations.

Embodiment 3A, The method of embodiment 1 or 2, wherein the microdose of the sodium channel blocker is at least about 10 milligrams in a single inhalation or multiple inhalations.

Embodiment 4. The method of any one of embodiments 1 to 3, wherein the microdose of the sodium channel blocker is up to 1000 micrograms in a single inhalation or multiple inhalations.

Embodiment 4A. The method of any one of embodiments 1 to 3, wherein the microdose of the sodium channel blocker is up to 10 milligrams in a single inhalation or multiple inhalations.

Embodiment 5. The method of any one of embodiments 1 to 4, wherein the microdose of the sodium channel blocker elicits an ECG change.

Embodiment 6. The method of any one of embodiments 1 to 5, wherein the microdose of the sodium channel blocker unmasks an ECG phenotype of BrS.

Embodiment 7. The method of embodiment 6, wherein the ECG phenotype of BrS is a Type 1, Type 2, or Type 3 BrS.

Embodiment 8. The method of any one of embodiments 5 to 7, wherein the microdose of the sodium channel blocker elicits an ECG change that prolongs a QRS interval.

Embodiment 9. The method of embodiment 8, wherein the QRS interval is prolonged in a manner that a J-wave amplitude is >2 mm.

Embodiment 10. The method of any one of embodiments 5 to 9, wherein the microdose of the sodium channel blocker elicits an ECG change on a T-wave morphology.

Embodiment 11. The method of any one of embodiments 5 to 10, wherein the microdose of the sodium channel blocker elicits an ECG change on a ST-T wave configuration.

Embodiment 12. The method of embodiment 11, wherein the ST-T wave configuration is coved shaped or saddleback shaped.

Embodiment 13. The method of any one of embodiments 5 to 12, wherein the microdose of the sodium channel blocker elicits an ECG change on a ST segment terminal portion.

Embodiment 14. The method of embodiment 13, wherein the ST segment terminal portion is gradually descending, elevated to be <1 mm, or elevated to be >1 mm.

Embodiment 15. The method of any one of embodiments 1 to 14, wherein the delivering of the microdose of the sodium channel blocker is repeated at least once to confirm the presence or absence of BrS.

Embodiment 16. The method of embodiment 15, wherein the delivering of the microdose of the sodium channel blocker is repeated two to five times to confirm the presence or absence of BrS.

Embodiment 17. The method of any one of embodiments 1 to 16, wherein the administering a microdose of a sodium channel blocker is done in a hospital or physician clinic setting as an outpatient.

Embodiment 18. The method of any one of embodiments 1 to 17, wherein the sodium channel blocker is a Class I anti-arrhythmic sodium channel blocker.

Embodiment 19. The method of embodiment 18, wherein the sodium channel blocker is a Class Ic anti-arrhythmic sodium channel blocker.

Embodiment 20. The method of embodiment 18, wherein the sodium channel blocker is flecainide.

Embodiment 21. The method of embodiment 18, wherein the sodium channel blocker is ajmaline.

Embodiment 22. The method of embodiment 18, wherein the sodium channel blocker is pilsicainide.

### EXAMPLES

The present invention will be further illustrated by way of the following Examples. These examples are non-limiting and do not restrict the scope of the invention. Unless stated otherwise, all percentages, parts, etc. presented in the examples are by weight.

### Example 1. Diagnosis of Brugada Syndrome Via Inhalation of Microdose of Flecainide

This example describes a diagnostic procedure for Brugada syndrome in a patient.

The patient, at the age of 30-50 years old, is subject to diagnosis of Brugada syndrome because the patient has a family history of sudden cardiac death, has been documented ventricular fibrillation before, has experienced self-terminating polymorphic ventricular tachycardia before, has a family member diagnosed as having coved-type ECG pattern, has electrophysiologic inducibility, or has syncope or nocturnal agonal respiration.

The patient is admitted at a hospital for the diagnostic test and attended to by an ECG technician and an attending physician. At the beginning of the test, the patient is provided by a healthcare provider (e.g., the ECG technician, the physician, or a nurse) with a kit containing a jet nebulizer, a vial of flecainide acetate solution at 500 microgram/mL, and a written instruction sheet with instructions on how to apply the flecainide acetate solution in the nebulizer, and how to manipulate the nebulizer for inhalation of 500 micrograms of flecainide. The patient is given oral instructions on how to self-administer the flecainide aerosol as well as given sufficient time to read the instruction sheet provided in the kit. Once the patient acknowledges understanding of the administration procedure, the ECG technician sets up ECG monitoring equipment on the patient and makes sure the ECG of the patient is being accurately and continuously monitored. Then, the patient is given instruction to start the administration of flecainide. Both the ECG technician and the attending physician keep close monitoring of the ECG of the patient during and for at least 2 hours after the administration of flecainide. If during the administration, the ECG phenotype of Brugada syndrome is unmarked, for instance, the appearance of type Brugada syndrome ECG pattern, or if the QRS interval is widened significantly, the physician will instruct the patient to stop the inhalation immediately.

The physician assesses the ECG pattern across the time before, during, and after the flecainide administration, and determines whether or not there is an ECG pattern indicative of Brugada, syndrome during or after the flecainide administration. For instance, if a type I Brugada syndrome ECG pattern appears, the physician will consider diagnosing the patient as having Brugada syndrome.

### Example 2. Pharmacokinetics Analysis of Inhalation Versus Intravenous Administration of Flecainide

This example describes pharmacokinetics (PK) analysis performed during a human clinical trial (FLE-001) in which inhalation administration of flecainide was tested for safety and compared against intravenous administration of flecainide.

This was an open label non-randomized crossover in a cohort of 6 evaluable healthy adult volunteers. This part of the study consisted of two periods with each subject receiving a total of 2 doses of flecainide, one dose in each period. In Period 1, 3 subjects received flecainide acetate solution by inhalation at the dose level of 30 mg estimated lung total dose (eTLD). and 3 subjects received a single dose of 2 mg/kg (or 150 mg, whichever is less) via a 10 min intravenous (IV) infusion of flecainide (Tambocor™ Injection; approved and used in clinical practice in Australia). In Period 2, the subjects who received flecainide inhalation solution in Period 1 now received a single dose of IV flecainide (2 mg/kg or 150 mg, whichever is less, via a 10 min infusion), while the subjects who received IV flecainide in Period 1 now received flecainide inhalation solution (30 mg eTLD). It was found that the baseline (pre-dose) values of HR, Systolic BP and Diastolic BP and, PR and QRS interval durations for Period 1 and Period 2 prior to dosing are near identical, which is consistent with the expectation from a cross-over design study. The interpretation is that there was no carry over effect of treatment or other changes in the subjects' vital signs and ECG intervals between the two periods.

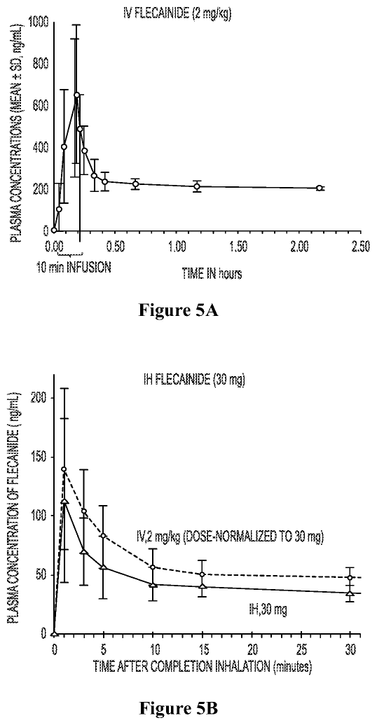

In the 6 subjects of this cohort, the venous plasma concentration-time curves for inhaled flecainide (30 mg eTLD) were similar to those for flecainide administered via IV infusion (2 mg/kg; FIGS. 5A and 5B). Peak plasma concentrations of flecainide (Cmax) following intravenous administration and inhalation were 749±308 and 120+70 ng/ml, respectively. The time to Cmax (Tmax) for intravenous infusion was between 1 and 60 minutes after the end of 10 min infusion, and ≤1 min post-inhalation.

### Example 3. Analytical Model Involving Inhalation of Verapamil

Published pharmacokinetic and pharmacodynamic models show relationships between drug concentration in coronary blood and desired coronary effect. IV drug information was used from published literature. HARRISON et al., “Effect of Single Doses of Inhaled Lignocaine on FEV1 and Bronchial Reactivity in Asthma,” Respir Med., 12:1359-635 (December 1992). Inhaled drug information was simulated based on known properties of pulmonary small molecule absorption.

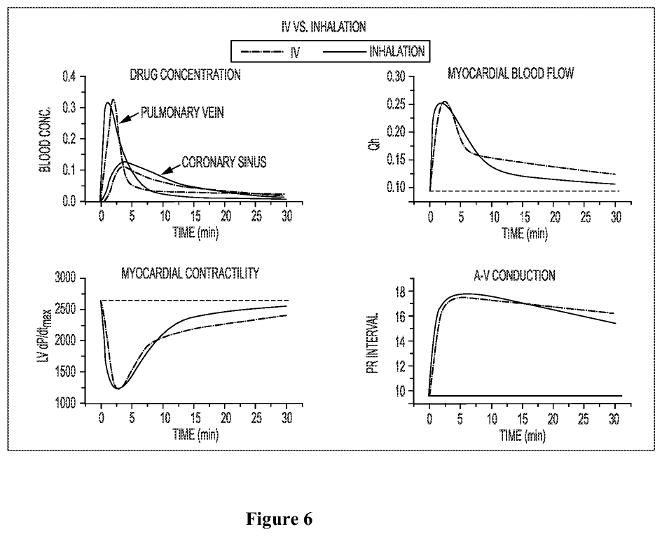

FIG. 6 shows the different time concentration profiles of drug administered via the IV and inhalation routes. Verapamil was selected as an example heart drug as it possesses both cardiac rate and rhythm control properties and is often used to rescue acute arrhythmia episodes (e.g., PSVT, paroxysmal supraventricular tachycardia).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of evaluating a subject in need thereof comprising: administering an aerosol of a sodium channel blocker to said subject, and assessing cardiac activity of said subject, wherein said cardiac activity is indicative of Brugada syndrome.

2. The method of claim 1, wherein said aerosol comprises a microdose of said sodium channel blocker per breath.

3. The method of claim 1 or 2, wherein said aerosol comprises at most about 1000 micrograms of said sodium channel blocker per breath.

4. The method of claim 3, wherein said aerosol comprises at least about 10 micrograms of said sodium channel blocker per breath.

5. The method of claim 3, wherein said aerosol comprises at least about 100 micrograms of said sodium channel blocker per breath.

6. The method of claim 3, wherein said aerosol comprises at least about 500 micrograms of said sodium channel blocker per breath.

7. The method of claim 1 or 2, wherein said aerosol comprises at most about 10 milligrams of said sodium channel blocker per breath.

8. The method of claim 1 or 2, herein said aerosol comprises at least about 20 milligrams of said sodium channel blocker per breath.

9. The method of claim 1 or 2, wherein said aerosol comprises at least about 50 milligrams of said sodium channel blocker per breath.

10. The method of claim 1 or 2. wherein said aerosol comprises at least about 100 milligrams of said sodium channel blocker per breath.

11. The method of claim 1 or 2, wherein said aerosol comprises about 100 micrograms to about 500 micrograms of said sodium channel blocker.

12. The method of any one of claims 1 to 11, wherein said aerosol comprises liquid droplets or dry powder, or an evaporative or condensation aerosol.

13. The method of any one of claims 1 to 12, further comprising producing said aerosol by a nebulizer, a metered dose inhaler, or a dry powder inhaler.

14. The method of claim 13, wherein said nebulizer is a vibrating mesh nebulizer or a jet nebulizer.

15. The method of claim 13, wherein said dry powder inhaler is an active dry powder inhaler or a passive dry powder inhaler.

16. The method of any one of claims 1 to 15, wherein said assessing cardiac activity of said subject comprises conducting an electrocardiogram (ECG) test on said subject.

17. The method of claim 16, wherein said ECG test is performed with a Holter monitor.

18. The method of claim 16, wherein said ECG test is a 12-lead ECG test.

19. The method of claim 16, wherein said ECG test measures at least one right precordial lead.

20. The method of claim 16, wherein said ECG test measures V1, V2, or V3 lead.

21. The method of any one of claims 1 to 20, wherein said administering of said sodium channel blocker elicits an ECG change in said subject.

22. The method of claim 21, wherein said ECG change appears within 60 minutes of said administering of said sodium channel blocker.

23. The method of claim 21, wherein said ECG change appears within 30 minutes of said administering of said sodium channel blocker.

24. The method of claim 21, wherein said ECG change appears within 10 minutes of said administering of said sodium channel blocker.

25. The method of claim 21, wherein said ECG change appears within 5 minutes of said administering of said sodium channel blocker.

26. The method of any one of claims 1 to 25, wherein said administering of said sodium channel blocker unmasks an ECG phenotype of Brugada syndrome in said subject.

27. The method of claim 26, wherein said ECG phenotype of Brugada syndrome is a Type 1, Type 2, or Type 3 Brugada syndrome ECG pattern.

28. The method of claim 26, wherein said ECG phenotype of Brugada syndrome comprises a J wave amplitude of >2 mm or 0.2 mV in more than one right precordial lead.

29. The method of claim 26, wherein said Type 1 Brugada syndrome ECG pattern comprises a negative T-wave following said J wave.

30. The method of claim 29, wherein said Type 1 Brugada syndrome ECG pattern comprises a coved ST-T configuration.

31. The method of claim 29 or 30, wherein said Type 1 Brugada syndrome ECG pattern comprises a descending terminal portion of ST segment.

32. The method of any one of claims 21 to 31, wherein said administering of said sodium channel blocker converts a normal ECG pattern without said sodium channel blocker to a Type 1, Type 2, or Type 3 Brugada syndrome ECG phenotype in said subject.

33. The method of any one of claims 21 to 31, wherein said administering of said sodium channel blocker converts a Type 2 Brugada syndrome ECG pattern without said sodium channel blocker to a Type 1 Brugada syndrome ECG pattern in said subject.

34. The method of claim 33, wherein said Type 2 Brugada syndrome ECG pattern comprises a J wave amplitude of >2 mm or 0.2 mV in more than one right precordial lead.

35. The method of claim 34, wherein said Type 2 Brugada syndrome ECG pattern comprises a positive or biphasic T-wave following said J wave.

36. The method of any one of claims 33 to 35, wherein said Type 2 Brugada syndrome ECG pattern comprises a saddleback ST-T configuration.

37. The method of any one of claims 33 to 36, wherein said Type 2 Brugada syndrome ECG pattern comprises a terminal portion of ST Segment that is elevated for at least about 1 mm or 0.1 mV.

38. The method of any one of claims 21 to 31, wherein administering of said sodium channel blocker converts a Type 3 Brugada syndrome ECG pattern without said sodium channel blocker to a Type 1 Brugada syndrome ECG pattern in said subject.

39. The method of claim 38, wherein said Type 3 Brugada syndrome ECG pattern comprises a J wave amplitude of >2 mm in more than one right precordial lead.

40. The method of claim 38, wherein said Type 3 Brugada syndrome ECG pattern comprises a positive T-wave following said J wave.

41. The method of any one of claims 38 to 40, wherein said Type 3 Brugada syndrome ECG pattern comprises a saddleback ST-T configuration.

42. The method of any one of claims 38 to 41, wherein said Type 3 Brugada syndrome ECG pattern comprises a terminal portion of ST Segment that is elevated for less than 1 mm or 0.1 mV.

43. The method of any one of claims 1 to 42, wherein said administering of said aerosol of said sodium channel blocker is repeated at least once to confirm a presence or absence of Brugada syndrome.

44. The method of claim 43, wherein said administering of said aerosol of said sodium channel blocker is repeated two to five times to confirm the presence or absence of Brugada syndrome.

45. The method of any one of claims 1 to 44, wherein said administering of said aerosol of said sodium channel blocker is performed in a hospital or a physician clinic setting.

46. The method of any one of claims 1 to 45, wherein said sodium channel blocker is a Class I antiarrhythmic agent.

47. The method of claim 46, wherein said sodium channel blocker is a Class Ic anti-arrhythmic agent.

48. The method of any one of claims 1 to 45, wherein said sodium channel blocker comprises flecainide or salt thereof.

49. The method of any one of claims 1 to 45, wherein said sodium channel blocker comprises flecainide acetate.

50. The method of any one of claims 1 to 45, wherein said sodium channel blocker is selected from the group consisting of: ajmaline, pilsicainide, flecainide, procainamide, salt and solvate thereof.

51. The method of any one of claims 1 to 50, wherein said subject has one or more of the following: (a) documented ventricular fibrillation; (b) self-terminating polymorphic ventricular tachycardia; (c) a family history of sudden cardiac death; (d) coved-type ECGs in family members; (d) electrophysiologic inducibility; or (e) syncope or nocturnal agonal respiration.

52. The method of any one of claims 1 to 51, wherein said subject has one or more genetic mutations associated with Brugada syndrome.

53. The method of any one of claims 1 to 52, further comprising performing genetic testing of said subject's genome for one or more genetic mutations associated with Brugada syndrome.

54. A unit dose comprising: a composition that comprises a sodium channel blocker in a microdose that is sufficient to elicit an ECG change that unmasks an ECG phenotype of Brugada syndrome in a subject, and a pharmaceutically acceptable excipient.

55. The unit dose of claim 54, wherein said composition comprises a solution.

56. The unit dose of claim 55, wherein said composition comprises an aqueous solution.

57. The unit dose of claim 55, wherein said composition comprises a non-aqueous solution.

58. The unit dose of any one of claims 54 to 57, wherein said composition comprises a pH buffer.

59. The unit dose of claim 58, wherein said composition comprises a pH buffer selected from the group consisting of: citrate, phosphate, phthalate, acetate, and lactate.

60. The unit dose of claim 54, wherein said composition consists essentially of said sodium channel blocker and water.

61. The unit dose of claim 54, wherein said composition consists essentially of said sodium channel blocker, water, and a pH buffer.

62. The unit dose of any one of claims 54 to 61, wherein the composition has a pH ranging from 3.5 to 8.0.

63. The unit dose of any one of claims 54 to 62, wherein said sodium channel blocker comprises a class Ic antiarrhythmic.

64. The unit dose of any one of claims 54 to 62, wherein said sodium channel blocker is selected from the group consisting of ajmaline, pilsicainide, flecainide, procainamide, salt and solvate thereof.

65. The unit dose of any one of claims 54 to 64, comprising at most about 1000 micrograms of said sodium channel blocker.

66. The unit dose of claim 65. comprising at least about 10 micrograms of said sodium channel blocker.

67. The unit dose of claim 65, comprising at least about 100 micrograms of said sodium channel blocker.

68. The unit dose of claim 65, comprising at least about 500 micrograms of said sodium channel blocker.

69. The unit dose of any one of claims 54 to 64, comprising about 100 micrograms to about 500 micrograms of said sodium channel blocker.

70. The unit dose of any one of claims 54 to 69, comprising a unit dose receptacle that contains the composition.

71. A kit comprising: the unit dose of any one of claims 54 to 70, and instructions for use of the unit dose for evaluating a presence or absence of Brugada syndrome in a subject in need thereof.

72. An aerosol comprising particles having a mass median aerodynamic diameter less than 10 μm, wherein said particles comprise: a sodium channel blocker in a microdose that is sufficient to elicit an ECG change that unmasks an ECG phenotype of Brugada syndrome in a subject, and a pharmaceutically acceptable excipient.

73. The aerosol of claim 72. wherein said particles comprise a nebulized solution.

74. The aerosol of claim 72, wherein said particles comprise a nebulized aqueous solution.

75. The aerosol of any one of claims 72 to 74, wherein the particles comprise a pH buffer.

76. The aerosol of any one of claims 72 to 74, wherein said particles comprise a pH buffer selected from the group consisting of: citrate, phosphate, phthalate, acetate, and lactate.

77. The aerosol of claim 72, wherein said particles consist essentially of said sodium channel blocker and water.

78. The aerosol of claim 72, wherein said particles consist essentially of said sodium channel blocker, water, and a pH buffer.

79. The aerosol of any one of claims 72 to 78, wherein said particles have a pH ranging from 3.5 to 8.0.

80. The aerosol of any one of claims 72 to 79, wherein said sodium channel blocker comprises a class Ic antiarrhythmic.

81. The aerosol of any one of claims 72 to 80, wherein said sodium channel blocker is selected from the group consisting of: ajmaline, pilsicainide, flecainide, procainamide, salt and solvate thereof.

82. The aerosol of any one of claims 72 to 81, comprising at most about 1000 micrograms of said sodium channel blocker.

83. The aerosol of claim 82, comprising at least about 10 micrograms of said sodium channel blocker.

84. The aerosol of claim 83, comprising at least about 100 micrograms of said sodium channel blocker.

85. The aerosol of claim 83, comprising at least about 500 micrograms of said sodium channel blocker.

86. The aerosol of any one of claims 72 to 81, comprising about 100 micrograms to about 500 micrograms of said sodium channel blocker.

87. A kit, comprising: a container containing a sodium channel blocker in a microdose that is sufficient to elicit an ECG change that unmasks an ECG phenotype of Brugada syndrome in a subject; and an aerosolization device.

88. The kit of claim 87, wherein the aerosolization device comprises a nebulizer.

89. The kit of claim 87, wherein the aerosolization device comprises a vibrating mesh nebulizer or a jet nebulizer.

90. The kit of claim 87, wherein the aerosolization device comprises a dry powder inhaler.

91. The kit of claim 87, wherein the aerosolization device comprises an active dry powder inhaler or a passive dry powder inhaler,

92. The kit of claim 87, wherein the aerosolization device comprises a metered dose inhaler.

93. The kit of any one of claims 87 to 92, wherein said sodium channel blocker comprises a class Ic antiarrhythmic.

94. The kit of any one of claims 87 to 92, wherein said sodium channel blocker is selected from the group consisting of: ajmaline, pilsicainide, flecainide, procainamide, salt and solvate thereof.

95. The kit of any one of claims 87 to 94, wherein said container comprises at most about 1000 micrograms of said sodium channel blocker.

96. The kit of claim 95, wherein said container comprises at least about 10 micrograms of said sodium channel blocker.

97. The kit of claim 95, wherein said container comprises at least about 100 micrograms of said sodium channel blocker.

98. The kit of claim 95, wherein said container comprises at least about 500 micrograms of said sodium channel blocker.

99. The kit of any one of claims 87 to 94, wherein said container comprises about 100 micrograms to about 500 micrograms of said sodium channel blocker.
